(19) United States
(12) Patent Application Publication
(10) Pub. No.: US 2006/0200232 A1
(43) Pub. Date: Sep. 7, 2006

(54) Nanofibrous materials as drug, protein, or genetic release vehicles

(76) Inventors: Matthew D. Phaneuf, Ashland, MA (US); Philip J. Brown, Williamston, SC (US); Martin J. Bide, South Kingstown, RI (US)

Correspondence Address: DAVID PRASHKER, 8 CHATEAU HEIGHTS, MAGNOLIA, MA, 01930, US

(21) Appl. No.: 11/366,165

(22) Filed: Mar. 2, 2006

Related U.S. Application Data
(63) Continuation-in-part of application No. 11/211,935, filed on Aug. 25, 2005.
(60) Provisional application No. 60/658,438, filed on Mar. 4, 2005.

(51) Int. Cl. A61F 2/06; B32B 27/12; D04H 13/00; B29C 47/00

(52) U.S. Cl. 623/1.42; 424/423; 264/465; 442/340; 442/121; 442/123

(57) ABSTRACT

The present invention is a bioactive, nanofibrous material construct which is manufactured using an unique electrospinning perfusion methodology. One preferred embodiment provides a nanofibrous biocomposite material formed as a discrete textile fabric from a prepared liquid admixture of (i) a biodurable synthetic polymer; (ii) a biologically active agent; and (iii) a liquid organic carrier. The prepared liquid admixture and fluid blending of diverse matter is employed in a novel electrospinning perfusion process to form an agent-releasing nanofibrous fabric, which in turn, can serve as the antecedent precursor and tangible workpiece for subsequently making the desired medical article or device suitable for use in-vivo. As the fabric is generated as a discrete article in either tubular or flat sheet form, one or more of the pre-chosen biologically-active agents will have become non-permanently immobilized and releaseably attached to the nanofibrous material of the fabric. These non-permanently immobilized biologically-active agents are chemical compounds which retain their recognized biological activity both before and after becoming non-permanently bound to the formed textile material; and will become subsequently released in-situ as discrete freely mobile agents from the fabric upon uptake of water from the ambient environment. Accordingly, the agent-releasing nanofibrous fabric is very suitable for inclusion and use in-vivo as a clinical/therapeutic medical article or device.

### PRIORITY CLAIM

The present invention was first filed on Mar. 4^{th}, 2005 as U.S. Provisional Patent Application No. 60/658,438. The priority and legal benefit of this first filing is expressly claimed.

### CROSS-REFERENCE

The present application is a Continuation-In-Part of U.S. patent application Ser. No. 11/211,935 filed Aug. 25, 2005 entitled “Nanofibrous Biocomposite Prosthetic Vascular Graft”. The legal benefit of this earlier-filed Non-Provisional U.S. patent application is expressly claimed.

### FIELD OF THE INVENTION

The present invention is concerned generally with improvements in biocomposite materials able to function as vehicles for the in-situ delivery and release of a diverse variety of biologically active agents; and is specifically directed to the manufacture and use of nanofibrous materials and fabricated composites comprised of fibers which will provide a combination of specific physical properties (such as biocompatibility, durability, compactness, and ease of application) and particular biologically active attributes (such as infection-resistance, anti-thrombin effects, growth promoting capabilities, growth inhibition capacities, analgesic effects and antimicrobial characteristics).

The instant invention provides a variety of formed fabric materials, articles, and devices suitable for the in-situ delivery of many different active agents; offers a wide range of fabricated nanofibrous textiles having varying and diverse individual biologic properties, or combinations thereof; and provides medical products which are resistant to breakage and tearing as well as demonstrate a specifically desired localized effect such as resistance to infection—properties which will aid in reducing both the morbidity and mortality of a person afflicted with an injury or ailment.

### BACKGROUND OF THE INVENTION

### Part I: Overall Medical Considerations

There are over 13 million medical articles and devices utilized annually in the United States for prophylactic and/or therapeutic treatment. These items range in sophistication from simple devices such as hernia repair mesh, wound dressings and catheter cuffs—to more complex implantable devices such as the total implantable heart, left ventricular assist devices and prosthetic arterial grafts. Although utilization of these medical articles and devices has improved the health and quality of life for the patient population as a whole, the in-vivo application of all such medical implements are prone to two major kinds of complications: infection and incomplete/non-specific cellular healing.

In general, regardless of the particular causative agent, infection remains one of the major complications associated with utilizing biomaterials, with the clinical infection occurring at either acute or delayed time periods after in-vivo use or implantation of the medical article or device. Today, surgical site infections account for approximately 14-16% of the 2.4-million nosocomial infections in the United States, and result in an increased patient morbidity and mortality. The inherent bulk properties of various biomaterials that comprise these articles and devices typically provide a milieu for initial bacterial/fungus adhesion with subsequent biofilm production and growth.

Similarly, unregulated cellular growth affects various medical devices such as stents and vascular grafts. Occlusion rates for diseased blood vessels after placement of a bare metallic stent (restenosis) have been reported as high as 27%, a significant problem based on the 1.1 million stents annually implanted. Moreover, since the currently available biomaterials in these medical articles and devices are typically comprised of foreign polymeric compounds, these biomaterials do not emulate the multitude of dynamic biologic and healing processes that occur in normal tissue; and consequently, the cellular components normally present within native living tissue are not available for controlling and/or regulating the reparative process. Thus, the search continues today for novel biomaterials (such as drug releasing biomaterials) that would direct or enhance some of the normal healing processes of native tissue, and would decrease patient morbidity and mortality rates.

One particular example of the broad need for novel drug releasing biomaterials is the treatment of traumatic injury. Regardless of whether the trauma is caused by a motor vehicle accident, pedestrian accident, accidental firearm discharge, recreational accident, criminal act, terrorist act or battlefield conditions, medical treatment of traumatic injury consistently results in significant rates of human morbidity and mortality. Thus, in 2002 alone, over 400,000 trauma cases were reported in the United States, with some 148,000 Americans dying each year. Of these mortalities, 40% have been attributed to uncontrolled bleeding at the trauma site; and overall, traumatic injuries have resulted in a total cost of $260 billion to the healthcare system, thereby accounting for 12% of all medical spending.

### Part II: The Two Major Kinds Of Medical Complications

Any penetration of the human body carries with it the risk of potential infection by microbes. This risk pertains particularly to traumatic wounds incurred by accident or negligence; to wound treatment procedures which utilize a wide range of materials for closure; and to the different kinds of articles used for skin penetrations and/or body wounds. In addition, there are also over 13 million medical devices which are surgically implanted in-vivo for prophylactic treatment or for therapeutic treatment of clinically diagnosed diseases, disorders, and pathological conditions in human patients annually in the United States alone.

Although utilization of these therapeutic/prophylactic treatments has markedly improved the overall health and quality of life for all persons, and especially an aging patient population, all such medical articles, manufactures, and devices are commonly susceptible to and routinely suffer from two kinds (or categories) of major complications. These are: (A) microbial infections; and (B) incomplete/non-specific cellular healing of the surrounding tissues. Each of these major complications is summarily reviewed below

### A. Microbial Infections

Infection, whether caused by viruses, bacteria or fungi, remains as one of the major complications associated with utilizing therapeutic biomaterials, and typically occur at either cute or delayed time periods after in-vivo use or implantation of the material or device. Surgical site infections account for approximately 14-16% of the 2.4-million nosocomial infections in the United States, and result in an increased patient morbidity and mortality.

Infection therefore remains one of the major complications associated with utilizing biomaterials, whether employed in a percutaneous or implantable fashion. Inoculation of the biomaterial presumably occurs at the time of operation or as a result of transient bacteremia/fungus in the immediate post-operative period. The intrinsic bulk properties of the various conventionally known biomaterials that typically comprise these devices provide a rich milieu for initial bacterial/fungus adhesion and cause subsequent biofilm production and growth. Moreover, perioperative parental antibiotics or antifungal agents often fail to permeate the avascular spaces immediately around biomaterials and the carbohydrate-rich bacterial biofilm once pathogens have adhered.

### Efforts To Combat Infections

Antimicrobial Agents

The rational use of antimicrobial agents against infection has been advocated generally; a Id such use has been previously reviewed in detail within the medical literature [see for example, Rodgers, K. G., Emer. Med. Clin. N. Am. 10: 753 (1992)]. Similarly, the major concerns regarding the ever-growing incidence of infections resulting from the use of biomedical articles and devices containing textiles, fabrics or fibers—despite recent advances in sterile procedures used in the clinical/surgical setting—have been recognized and considered to be of primary importance [see for example, the FDA/EPA/CDC/AAMI joint conference in Proceedings, Infection Control Symposium: Influence Of Medical Device Design, U.S. Dept. of Health and Human Services, Bethesda, Md., January 1995]. Moreover, the use of antibiotics and antivirals, as well as the development of mechanisms for delivering antimicrobial agents generally (particularly via slow-release delivery systems over time) to prevent or reduce severity of infection for implanted biodegradable materials has become prominent [see for example, Sasmor et al., J Vasc. Sur. 14: 521 (1993)]. All of these developments and considerations lead to the same conclusion: Infection, with or without the use of antibiotics, must be prevented or be controlled for all textile fiber containing materials regardless of clinical need or medical purpose.

Strategies

Numerous strategies have been proposed and attempted to be implemented in order to create an infection-preventing surface for biomaterials. Much of this effort has been directed at surgically implantable textiles and in-vivo engraftable articles. However, these efforts to reduce and to combat surgical infections in-vivo are merely a representative portion of the greater problem as a whole directed towards biomaterials comprised of fibrous matter which are able to prevent and interdict infections generally—i.e., without regard to whether or not the potential infection is airborne, topical, percutaneous or subcutaneous, humoral, and organ or tissue specific.

For example, a variety of different chelating agents have been evaluated for use as a release system for antibiotics from a biomaterial surface. One favored approach has been the ionic binding of antibiotics by surfactants. Cationic surfactants (such as tridodecylmethyl ammonium chloride and benzalkonium chloride) were sorbed at the anionic surface potential of a polymeric material, thereby achieving a weak adhesion of anionic antibiotics to the surface of the polymer [see for example: Harvey et al., Ann. Surg. 194: 642 (1981); Harvey et al., Surgery 92: 504 (1982); Harvey et al., Am. J. Surg. 147: 205 (1984); Shue et al., J. Vasc. Surg. 8: 600 (1988); and Webb et al., J. Vasc. Surg. 4: 16 (1956)]. The surfactant immobilized antibiotic subsequently was released into mobile form upon contact with blood.

Silver was also examined as a release system for various antibiotics from textile surfaces. Silver was applied either as a chelating agent [see for example: Modak et al., Surg. Gynecol. Obstet. 164: 143 (1987); Benvenisty et al., J. Surg. Res. 44: 1 (1988); and White et al., J. Vasc. Surg. 1: 372 (1984)], or alone in metallic form, for its antimicrobial properties.

Another favored approach has employed various binding agents in order to create localized concentrations of an antibiotic on the article's surface. These binding agents, typically a protein or a synthetic-based substance, were embedded within the biomaterial matrix, thereby either “trapping” or ionically binding with the antibiotic of choice. In this manner, the basement membrane protein collagen has often served as a binding agent and as a release system for rifampin, demonstrated to have antimicrobial efficacy in a bacteremic challenge dog model [Krajicek et al., J. Cardiovasc. Surg. 10: 453 (1969)] as well as in early European clinical trials [Goeau-Brissonniere, O., J. Mal. Vasc. 21: 146 (1996); Strachan et al., Eur. J. Vasc. Surg. 5: 627 (1991)].

Similarly, fibrin, present either as a glue or as a factor in pre-clotted blood, has been utilized as a binding agent for the immobilization of various antibiotics, including gentamycin, rifampin and tobramycin [see for example, Haverich et al., J. Vasc. Surg. 14: 187 (1992); McDougal et al., J. Vasc. Surg. 4: 5 (1986); Powell et al., Surgery 94: 765 (1983); Greco et al., J. Biomed. Mater. Res. 25: 39 (1991)].

Furthermore, Levofloxacin (itself a quinolone, a synthetic analog of nalidixic acid) has been incorporated in an albumin matrix and gelatin has been used as the release system for the antibiotics rifampin and vancomycin, with animal studies also showing efficacy in acute bacteremic challenges [see for example, Muhl et al., Ann. Vasc. Surg. 10: 244 (1996); Sandelic et al., Cardiovasc. Surg. 4: 389 (1990)].

In addition to the foregoing, a variety of synthetic binding agents have also been evaluated for antibiotic release as a replacement for the naturally occurring protein binders. Some synthetic binders were incorporated directly into the biomaterial matrix (in a similar fashion to the protein binders) which permitted a sustained release of a selected antibiotic over time [see Shenk et al., J. Surg. Res. 47: 487 (1989)]. Recent techniques also have utilized these types of synthetic binder materials as a scaffolding to bind antibiotics covalently to the biomaterial surface [see Suzuki et al., ASAIO J. 43: M854 (1997)]. Release of the antimicrobial agent was controlled by bacterial adhesion to the surface, which resulted in antibiotic cleavage and release. This mechanism of activity promotes “bacterial suicide” while maintaining antibiotic concentration, which is not needed to prevent infection, localized on the surface.

Other techniques have involved which incorporate the antibiotic either into the process of synthesizing the polymer [see Golomb et al., J. Biomed. Mater. Res. 25: 937 (1991); Whalen et al., ASAIO J. 43: M842 (1997); or embed the antibiotic directly into the interstices of the material [Okahara et al., Eur. J. Vasc. Endovasc. Surg. 9: 408 (1995)].

Recognized Drawbacks and Complications

It will be recognized and appreciated also that there are several serious drawbacks and undesirable complications in effect for each of these individual antibiotic immobilization strategies. For the approach using chelation agents, 50% of the antibiotic has been shown to elute from the graft surface within 48 hours, with less than 5% antibiotic remaining after three weeks [see Greco et al., Arch. Surg. 120: 71 (1985)]. While this degree of antibiotic coverage is adequate for small localized contaminations, it is clear that large infectious inoculums are not addressed.

In contrast, with the approach using binding agents, antibiotic release often is quite varied and will depend on the rate of binder degradation or binder release from a surface which is under high shear stress from blood flow. Comparably, both types of surface modifications rely on exogenous matter which may affect the overall properties of the textile surface, either by releasing toxic moieties or by promoting thrombogenesis. Thus, these potential complications have accentuated the need to create an infection-preventing textile fabric surface which is devoid of exogenous matter such as binding agents.

Prevailing Practices

Currently, drug delivery from a majority of implantable medical devices such as stents is achieved via the coating/sealing of the device with a prepared polymer composition which serves as a drug reservoir. There are several potential problems with utilizing this system in that: (1) polymer coating onto the device can be inconsistent, resulting in areas with minimum/no localized drug release; (2) polymer coating efficiency can be limited based on the device design or composition of the base material; (3) drug release is dependent on degradation of the polymer reservoir, resulting in inconsistent drug release; and (4) application of the exogenous polymer can have adverse effects on tissue/organ healing or upon the biocompatibility (i.e. increasing thrombogenecity) of the original implant.

### B. Incomplete/Non-Specific Cellular Healing Of Surrounding Tissues

Unregulated microbial growth will markedly affect any and all medical devices implanted in-vivo, such as stents and vascular grafts. The occlusion rates for diseased blood vessels after the in-vivo placement of a bare metallic stent (i.e., restenosis) have been reported as high as 27% of patients, a significant problem based on the approximately 1.1 million stents annually implanted. Also, since biocomposite materials are often comprised at least in part of metal and/or foreign polymeric materials, the cellular moieties and agents normally present within the native tissue of the patient are not present for controlling and/or regulating the reparative process. A commonality among this category of complications is that the currently available biomaterials do not emulate the multitude of dynamic biologic and reparative processes that typically occur as part of normal tissue healing.

### Conventional Means For Preventing Unregulated Microbial Growth

A Seeded and In-Situ Grown Endothelial Cell Layer

One of the conventionally proposed mechanisms to enhance biocompatibility of an implantable material is to develop a uniform endothelial cellular layer on the surface of the biomaterial. In theory, these layered cells, while providing structural stability via a material incorporation into the tissue surrounding the implant, would also serve to maintain hemostasis, to prevent infection, and to synthesize bioactive mediators. However, this type of in-vivo developed, layered endothelial cellular incorporation does not often occur in actuality or fact, thereby predisposing the implanted biomaterials to infection and thrombosis.

Clearly, the failure of appropriate cell type growth development to occur in-situ for these biomaterials significantly limits their use in-vivo. This unfortunate complication is evident both instent deployment as well as with the implantation of vascular grafts. Unregulated cellular growth often occurs within an endovascular stent, and at the material/artery interface for prosthetic vascular grafts; and this event results in the inevitable narrowing of the blood vessel lumen, with subsequent occlusive thrombosis occurring routinely.

Use of Adhesive Proteins

Nevertheless, when implanting prosthetic grafts or endovascular stents in-vivo, cellular adhesion to biomaterials using cell-seeding techniques has been extensively employed. Adhesive proteins such as fibronectin, fibrinogen, vitronectin and collagen have been employed fir this purpose and have apparently served well in such graft seeding protocols. The cell attachment properties of these matrices can also be duplicated by short peptide sequences such as RGD—i.e., Arg-Gly-Asp. The use of these adhesive proteins, however, has several drawbacks. These include: (1) bacterial pathogens recognize and will bind to these peptide sequences; (2) non-endothelial cell lines also will bind to these sequences; (3) patients requiring a seeded cell material, such as for implanting a vascular graft, have few donor endothelial cells, and therefore such cells must be initially grown in culture; and (4) endothelial cell loss to shear forces from flowing blood remains a medically serious obstacle.

Use of Surface Modifications

Modification of the biomaterial surface has also been employed to modify the host's response to the implanted article, and can serve as an approach for improving cellular adherence. Those cells that can be seeded have been empirically shown to be able to attach to and grow well upon a variety of different protein substrates which have been previously coated onto the surface of the biomaterial. Bioactive oligopeptides and cell growth factors have each been immobilized onto the surfaces of various polymers and empirically demonstrated to effect cell adherence and growth. Additional studies have reported the incorporation of growth factors into a degradable protein mesh, which then resulted in the in-situ formation of capillaries into the mesh material.

Utilizing these reported techniques to incorporate growth factors onto the surface of a biocomposite material matrix, however, does have a number of particular limitations. These include the following problems: (1) the growth factor sometimes is rapidly released from the matrix; (2) any degradation of the underlying matrix re-exposes a potentially thrombogenic surface; (3) endothelialization of the biomaterial surface sometimes is not uniform; and (4) the release of non-endothelial specific growth factor is not confined to the biomaterial matrix, thereby exposing the “normal” distal artery to the growth factor.

### Part III: Electrospinning Of Polymers To Form Nanofibrous Materials

Electrospinning provides a technique for making nanofibrous material substrates. Several parameters are recognized as necessary and attributed to the successful formation of a nanofibrous material produced by electrostatic means. These include: (1) the magnitude of the electric potential in relation to the distance between the emitter and the collector as well as the discharge media; (2) the viscosity of the polymer solution as determined by molecular weight and/or percent solids of the solution; and (3) the surface tension at droplet surface as determined by solvent/polymer interaction. These parameters and factors build on the rapid development seen in electrospinning over a number of years, including those investigating the formation of electrospun tubular structures.

Electrospinning to produce nanoscale fibers, fabrications and textiles, however, is still a manufacturing technique in need of further development and refinement. Utilization of electrospinning as a technique to synthesize various nanofibrous materials from polymers such as polyurethane, polyvinyl alcohol (or “PVA”), poly (lactic glycolic) acid (or “PLGA”), nylon, and polyethylene oxide has been investigated for several decades (see for example Subbiah et al., “Electrospinning Of Nanofibers”, J. Applied Polymer Sci. 96:557-569 (2005).

While some synthesis processes have been established for the use of these polymer compounds, some of the major drawbacks to advancing these materials into a clinical and/or therapeutic setting have been the well established significantly low break point and tear strength of the fabricated materials. These physical properties are critical to the development of clinically-useful materials and surgically implantable devices, which will need to possess and demonstrate excellent suture retention, burst strength, break strength, tear strength and/or biodurability under in-vivo use circumstances. The lack of overall material strength has been one of the major obstacles for developing novel nanofibrous medical devices.

In addition, while inclusion of bioactive agents has been accomplished for several other polymers (such as polyurethane, PLGA, alginate and collagen), the electrospinning technique has not been realized for polyethylene terephthalate (“PET”), or “polyester” as understood generally in textile circles, until recently. Since then, Ma et al. was able to electrospin polyethylene terephthalate using a melt-spinning technology [see Ma Z, Kotaki M, Yong T, He W, Ramakrishna S., “Surface engineering of electrospun polyethylene terephthalate (PET) nanofibers towards development of a new material for blood vessel engineering”, Biomaterials 26:2527 (2005)]. However, the Ma et al. reported technique requires a surface modification in which formaldehyde and several cross-linkers were utilized post-spinning subsequently to incorporate gelatin, owing to the high temperatures employed in their manufacturing process. These modification procedures are and remain a major issue because of their high temperature requirements and the consequential failure of the protein (or other temperature sensitive agent) to maintain its characteristic biological activity throughout the material fabrication process. Additionally, the resulting material should also possess particular physical characteristics such as tensile strength, a prerequisite to creating a novel medical device.

Accordingly, despite all these developments to date, there remains a recognized and continuing need for further improvements in the making of medical devices and articles comprised of nanofibrous materials which would demonstrate adequate physical strength characteristics and durability as fabricated items, and which would serve as biomedical constructs formed of fibrous materials having demonstrable biologically active properties. All such improvements in the making and/or preparation of such nanofibrous materials and articles would be readily seen as a major advantage and outstanding benefit in the medical field.

### SUMMARY OF THE INVENTION

The present invention is a major advance in the development of biomedical materials, devices and constructs. Accordingly, the invention has multiple aspects, some of which may be defined as follows.

A first aspect provides an agent-releasing textile useful for the making of a medical article or device, said agent-releasing textile comprising:

a nanofibrous composite material comprised of at least one biodurable synthetic substance and fabricated as an elongated hollow tubular structure via an electrospinning perfusion process, said fabricated nanofibrous tubular structure having determinable inner and outer wall diameters, two open ends, and an internal lumen, and being biocompatible for the conveyance of fluid through its internal lumen; and

at least one pre-chosen biologically active agent having recognized and characteristic mediating properties which has been combined with said biodurable synthetic substance in liquid admixture and has become non-permanently immobilized into said fabricated nanofibrous tubular structure as a consequence of said electrospinning perfusion process, said non-permanently immobilized active agent being released from said nanofibrous tubular structure and delivered in-situ into the surrounding environment as mobile active agent after said nanofibrous tubular structure takes up fluid.

As second aspect of the invention provides an agent-releasing textile useful for the making of a medical article or device, said agent-releasing textile comprising:

a nanofibrous composite material comprised of at least one biodurable synthetic substance and fabricated as a flat sheet fabric via an electrospinning perfusion process, said flat sheet nanofibrous fabric having a determinable length, width, and depth and being biocompatible with the tissues and organs of a living subject; and

at least one pre-chosen biologically active agent having recognized and characteristic mediating properties which has been combined with said biodurable synthetic substance in liquid admixture and has become non-permanently immobilized into said fabricated flat sheet fabric as a consequence of said electrospinning perfusion process, said non-permanently immobilized active agent being released from said nanofibrous flat sheet fabric and delivered in-situ into the surrounding environment as mobile active agent after said nanofibrous flat sheet fabric takes up fluid.

A third aspect includes an electrospinning perfusion method for fabricating a flat sheet textile, said method comprising the steps of:

erecting an electrospinning perfusion assembly comprised of a rotating flat surface which can be set at a selected rotation speed, at least one perfusion instrument which can be set at a specified liquid flow rate, and an electrical coupling for controlling and coordinating the actions of said perfusion instrument upon said rotating flat surface;

preparing a fluid mixture comprised of at least one biodurable synthetic substance and an organic liquid carrier;

introducing said prepared fluid mixture to said perfusion instrument of said assembly;

perfusing said fluid admixture onto said rotating flat surface for a predetermined time such that a nanofibrous flat sheet textile fabric is fabricated, wherein said nanofibrous flat sheet textile fabric has a determinable length, width, and depth and is biocompatible with the tissues and organs of a living subject.

A fourth aspect provides an electrospinning perfusion method for fabricating an agent-releasing textile fabric, said method comprising the steps of:

erecting an electrospinning perfusion assembly comprised of a rotating flat surface which can be set at a selected rotation speed, at least one perfusion instrument which can be set at a specified liquid flow rate, and an electrical coupling for controlling and coordinating the actions of said perfusion instrument upon said rotating flat surface;

preparing a fluid mixture comprised of at least one biodurable synthetic substance, at least one pre-chosen biologically active agent having recognized and characteristic mediating properties, and an organic liquid carrier;

introducing said prepared fluid mixture to said perfusion instrument of said assembly;

perfusing said fluid admixture onto said rotating flat surface for a predetermined time such that a nanofibrous flat sheet textile fabric is fabricated, wherein said nanofibrous flat sheet textile fabric has a determinable length, width, and depth and said biologically active agent has become non-permanently immobilized upon said fabricated nanofibrous flat sheet textile fabric as a consequence of said perfusion, said non-permanently immobilized active agent being released from said nanofibrous flat sheet textile fabric and delivered in-situ into the surrounding environment as mobile active agent after said flat sheet fabric takes up fluid.

A fifth aspect provides an electrospinning perfusion method for fabricating an agent-releasing textile fabric, said method comprising the steps of:

erecting an electrospinning perfusion assembly comprised of a rotating mandrel which can be set at a selected rotation speed, at least one perfusion instrument which can be set at a specified liquid flow rate, and an electrical coupling for controlling and coordinating the actions of said perfusion instrument upon said rotating mandrel;

preparing a fluid mixture comprised of at least one biodurable synthetic substance, at least one pre-chosen biologically active agent having recognized and characteristic mediating properties, and an organic liquid carrier;

introducing said prepared fluid mixture to said perfusion instrument of said assembly;

perfusing said fluid admixture onto said rotating mandrel for a predetermined time such that a nanofibrous tubular textile is fabricated, wherein said nanofibrous tubular textile has determinable inner wall and outer wall diameter sizes, two open ends, and an internal lumen, and is biocompatible for the conveyance of fluid through its internal lumen, and said biologically active agent has become non-permanently immobilized upon said walls of said nanofibrous tubular textile as a consequence of said perfusion, said non-permanently immobilized active agent being released from said nanofibrous tubular textile and delivered in-situ into the surrounding environment as mobile active agent after said tubular textile takes up fluid.

### BRIEF DESCRIPTION OF THE FIGURES

The present invention may be more easily understood and more readily appreciated when taken into conjunction with the accompanying drawing, in which:

FIG. 1 is an illustration of the chemical structure of Ciprofloxacin;

FIG. 2 is an illustration of the chemical structure of Diflucan;

FIG. 3 is an illustration of the chemical structure of Paclitaxel;

FIG. 4 is a an illustration of the apparatus for performing the electrospinning methodology;

FIG. 5 is scanning electron microphotograph of a nPET (electrospun polyethylene terephthalate) textile segment showing the diameter size of the fibers within the nanofibrous material;

FIG. 6 is an overhead view of the UV illumination differences between nPET segments, nPET-Cipro segments, and nPET-Diflucan segments;

FIG. 7 is a graph showing the release profile of Cipro from nPET-Cipro segments over time;

FIG. 8 is a graph showing the release profile of Diflucan from nPET-Diflucan segments over time;

FIG. 9 is a an overhead view of the inhibitions zone against Staphylococcus aureus streaked onto agar plates;

FIG. 10 is a graph showing the antimicrobial activity of nPET-Cipro segments over time;

FIG. 11 is a graph showing the anti-fungal activity of nPET-Diflucan segments against varying concentrations of Candida albicans; and

FIG. 12 illustrates an overhead view of a flat sheet of electrospun textile fabric.

### DETAILED DESCRIPTION OF THE INVENTION

### I. The Subject Matter Of The Present Invention As A Whole

The present invention is a bioactive, nanofibrous material construct which is manufactured either in tubular or flat sheet form using an unique electrospinning perfusion methodology. One preferred embodiment provides a nanofibrous biocomposite material formed as a discrete textile fabric from a prepared liquid admixture of (i) a biodurable synthetic polymer; (ii) a biologically active agent; and (iii) a liquid organic carrier. The prepared liquid admixture and blending of diverse compositions is employed in a novel electrospinning perfusion process to form an agent-releasing textile comprised of nanofibrous material, which in turn, can serve as the antecedent precursor and tangible workpiece for subsequently making the desired medical article or device suitable for use in-vivo.

After the agent-releasing textile has been fabricated as a discrete article, one or more pre-chosen biologically-active agents will have become non-permanently immobilized and releaseably bound to the tangible nanofibrous material of the fabricated textile. These non-permanently immobilized biologically-active agents are well established chemical compounds which retain their recognized biological activity both before and after becoming impermanently (i.e., temporarily or reversibly) bound to the textile fabric; and will become subsequently released in-situ and directly delivered into the ambient environment as discrete mobile entities when the textile fabric takes up any fluid—i.e., any aqueous or organic based liquid. Accordingly, via the transitory immobilization of one or more biologically active molecules to the nanofibrous biocomposite material, the agent-releasing textile is very suitable for inclusion and use in-vivo as a clinical/therapeutic construct.

The present electrospinning perfusion method of making agent-releasing nanofibrous textiles provides several major advantages and desirable benefits to the commercial manufacturer as well as to the physician and surgeon. Among these are the following:

1. The manufacturing methodology comprising the present invention does not utilize any immersion techniques and does not require submerging the fabricated textile in any immersion baths, soaking tanks, or dipping pools for any purpose. Rather, the methodology preferably utilizes the unique technique of electrospinning perfusion as a manufacturing method in order to blend a synthetic substance and a biologically active agent of choice together as a fabricated textile.

2. The electrospinning perfusion method of manufacture yields a fabricated textile having particular characteristics. The fabricated textile is initially fashioned either as an elongated hollow tube having two discrete open tubular ends and fixed inner and outer wall diameters; or as a flat or planar sheet of nanofibrous fabric. In either format, the fabricated textile can be folded, or twisted, and otherwise manipulated to meet specific requirements of thickness, gauge, or deniers; and can also be cut, split, tailored, and conformed to meet particular shapes, configurations and patterns.

3. The fabricated textile is a nanofibrous material composite comprised of multiple fibers, has a determinable individual fiber thickness in or near the nanometer size range, and presents a discernible fiber organization and distribution pattern. These fabricated textiles provide and demonstrate excellent suture retention, burst strength, break strength, tear strength and/or biodurability.

4. The manufacturing method comprising the present invention employs limited heat and compression force to alter the exterior surface of the fabricated textile originally formed via the electrospinning perfusion technique. This exterior surface treatment portion of the manufacturing process is optional, but when employed, will produce a highly desirable crimped exterior surface over the entire linear length of the fabricated textile article. A notable feature of this exterior surface treatment procedure is that the inner diameter size (typically less than 1 mm to not greater than about 30 mm, but can vary from these particular parameters) of the fabricated textile remains constant and uniform, despite the effects of the limited heating and compression treatment of the textile exterior surface.

5. The biologically active agent of choice which is temporarily attached to the material substance of the fabricated textile (but which is released upon the uptake of liquid in-vitro and in-vivo as a freely mobile entity) will retain its characteristic biological activity both before and after being temporarily bound to the nanofibrous material. The attributes and properties associated with the biologically active agent of choice will co-exist with and be an integrated feature of the resulting textile article at the time it is utilized.

### Wording, Terminology, And Titles

Although many of the words, terms and titles employed herein are commonly used and conventionally understood within its traditional medical usage and scientific context, a summary description and definition is presented below for some phrases and wording as well as for particular names, designations, epithets or appellations. These descriptions and definitions are provided as an aid and guide to recognizing and appreciating the true variety and range of applications intended for inclusion within the scope of the present methodology.

To perfuse and a perfusion: The action and the act of causing a liquid or other fluid to pass across the external surfaces of, or to permeate through, the substance of a tangible entity or a configured physical construct. Perfusion of a liquid or fluid thus includes the alternative actions of: a sprinkling, pouring, or diffusing through or overlaying action; a covering, spreading, penetrating or saturating action (termed “suffusion”); a slow injection or other gradual introduction of fluid into a configured space or sized internal volume (termed “infusion”); and a passage across a surface or through a discrete surface or tangible thickness of matter, regardless of the mechanism or manner of transfer employed for such fluid passage.

To immerse and an immersion: The action and the act of dipping, plunging or sinking a discrete entity or tangible item completely such that it is entirely submerged within a quantity of liquid or a volume of fluid. Immersion of a discrete entity or tangible item also includes the alternative actions of: dunking, soaking, bathing, or flooding the entity within a liquid or fluid bath, tank, or pool; and the enveloping or burying of the tangible item in the liquid or fluid completely such that the item disappears from the surface and lies within the substance of the liquid or fluid matter.

Nonwoven fabric: A bonded or entangled web of material produced directly from fibers without first making yarns. The web of fibers is generally produced by carding, air-laying or wet-laying; and is subsequently bonded or entangled by heating, needle punching, water-jets (“spunlacing”), chemical glues, or by using chemical means. Those methods that combine web formation and bonding include melt blowing. The non-woven manufacturing process is typically used to yield light-weight, disposable fabrics and cloths.

Fabricated textile: An article of manufacture which is comprised, in whole or in part, of fibers arranged as a fabric. The fibers comprising the fabricated textile may be chosen from a diverse range of organic synthetics, prepared polymer compounds, or naturally-occurring matter. In general, the fabricated textile is often prepared as a cloth or fabric; and may comprise a single fiber film, or a single layer of fibrous matter; or exist as multiple and different deniers of fibers which are present in a range of varying thickness, dimensions, and configurations.

Agent-releasing textile: A fabricated textile comprising nanofibrous matter which has at least one biologically active agent immobilized onto and/or within the material substance of the textile; and which, upon wetting, is then able to release the biologically active agent in-situ and deliver it in a functional operative form into the adjacent local area or immediately surrounding environment. Such a prepared nanofibrous textile must provide and release at least one active chemical composition, compound, or molecule which is active, functional and operative either to influence and/or to initiate or cause a recognizable pharmacological effect or determinable physiological change in the living cells, tissues and organs of the host patient.

Aqueous mixture, liquid or fluid: By definition, any mixture, liquid or fluid which contains or comprises water in any meaningful quantity or degree. Although many other compositions, substances, or materials may exist within the mixture, fluid or liquid in a variety of physical states, the bulk or majority of volume for such fluids is water.

Organic liquid-miscible substance: By definition, any composition, compound, polymer material or matter in any physical state (i.e., gaseous, liquid or solid) that is capable of being mixed or combined with a liquid organic carrier. This term thus encompasses and includes within its meaning a variety of alternative conditions and physical states for any substance which is capable of: (i) being soluble or solubilized in any meaningful degree in a liquid organic solvent or an organic solvent blending; (ii) being mixed in any measurable quantity in an organic liquid or an organic fluid blending (whether or not a solution is formed); and (iii) being dispersed, or suspended, or carried in any quantity in an organic liquid or an organic fluid blending (whether or not a homogeneous suspension is formed).

Genetic material: By definition, any compound or substance comprised of two or more nucleic acids which are joined together to form a biologically-functional molecule. Nucleic acids typically are comprised of adenine, guanine, cytosine, thymine and uracil. Examples of such compounds comprising this class of substances are nucleotides, oligonucleotides, RNA (and its various forms), DNA, silencing RNA, as well as their sense and anti-sense formats.

### II. The Agent-Releasing Nanofibrous Textile And Its Role As An Antecedent In The Making of a Prepared Medical Article Or Device

The method of the present invention is directed in part to the making of an agent-releasing textile, an antecedent article of manufacture, which is then employed as a tangible workpiece to generate a subsequently prepared medical article or device suitable for use in-vivo.

The term “fabricated textile” has been defined in meaning above; and applies generally to any article, device, appliance, or construct which contains fibers, or is constituted of fibrous matter, or has as a component part or material substance comprised in whole or in part of discrete fibers. The broad and encompassing scope of this term is intentional; and is deemed to cover and apply to any and all textile-containing medical articles, devices, apparatus, appliances, instruments, and other tangible entities which are biocompatible with and/or can be surgically implantable into the tissues and organs of a living subject, human or animal.

In comparison, the term “agent-releasing textile” is defined and employed herein to identify those prepared nanofibrous material fabrics which upon use are able to release in-situ and deliver into the local or surrounding environment at least one chemical composition, compound, or molecule which is functional and operative to influence, and/or to alter, and/or to initiate or cause a particular pharmacological effect or a specific physiological change within the living cells, tissues and organs of the living host.

It will be appreciated that, after the agent-releasing nanofibrous textile has been manufactured and is present as a discrete entity, it can optionally serve as a tangible workpiece in combination with other items and additional components and hardware to yield the desired end product, a clinically or therapeutically useful “medical article or device”. Thus, regardless of its true chemical composition/formulation or the particular mode of construction, the initially formed ‘agent-releasing textile’ and the subsequently generated ‘medical article or device” are directly and intimately related; and thus share a number of specific qualities and characteristics in common. These mutually shared attributes include:
(i) Each agent-releasing textile is formed as an elongated hollow tube having a determinable overall tubular length and two open ends; has at least one internal lumen of determinable volume which is co-incidental and coextensive with the internal wall surface; and has at least one exterior wall surface which is co-incidental and co-extensive with the outer wall topography.
(ii) Each agent-releasing textile has a determinable length, girth and depth of non-perforated fibrous material which can be prepared to meet specific shapes, sizes and thicknesses of solid matter;
(iii) Each agent-releasing textile can be employed either as a configured tubular conduit whose internal lumen is usefully employed for the conveyance of fluids in-situ; or, alternatively, as a solid mass of nanofibrous material which achieves its intended purpose without regard to or actual use of the internal lumen then existing within the textile fabric.

By definitional requirement, the agent-releasing nanofibrous textile (optionally also the antecedent forerunner of each subsequently generated medical article or device) is a non-woven material comprised of discrete fibers. The nanofibrous composite material forming the textile fabric has been electronspun from a liquid admixture and blending in a liquid organic carrier of at least two different materials: a synthetic substance and a biologically active agent. This admixture of two diverse chemical compositions can be prepared in a wide range of varying ratios using a liquid organic carrier, followed by application of an electric current to create the biocomposite material

### A. The Chemical Formulation Of The Synthetic Fibers

To illustrate the range and variety of compositions deemed suitable for use as a blended mixture, a listing of suitable synthetic substances is presented by Table 1 below. It will be noted that the listing of Table 1 presents some exemplary synthetic substances long deemed suitable for use as synthetic fibers. To complete the description, Table 2 lists some of the typical and more commonly available organic liquids which can be usefully employed alone and/or in blends as the liquid carriers.

TABLE 1
Illustrative Synthetic Substances

Polymeric Fibers
polyethylene terephthalate;
polyurethane;
polyglycolic acid;
polyamides, including nylons and aramids;
polytetrafluoroethylene; and
mixtures of these substances.
Other synthetic fiber compositions
acetate;
triacetate;
acrylic;
modacrylic;
polypropylene; polyethylene, and other polyolefins;
saran.

TABLE 2
Representative Organic Liquid Carriers
Organic Liquid Carriers

Hexafluoroisopropanol;
Dimethylformamide;
Dimethylsulfoxide;
Acetonitrile;
Acetone;
Hexamethylphosphoric triamide;
N,N-diethylacetamine;
N-methylpyrrolidinone;
Ethanol.
4-methylmorpholine-N-oxide monohydrate

At least some of the fibers comprising the textile fabric will demonstrate a range of properties and characteristics, as follows.

1. The fibers constituting the agent-releasing textile (and the subsequently generated medical article or device) will have a demonstrable capacity to take up water and/or aqueous liquids and/or organic liquids and/or organic based liquids (with or without direct wetting of the fibrous material). The mode or mechanism of action by which organic and aqueous fluids are taken up by the fibers of the textile (and/or become wetted by the fluid) is technically insignificant and functionally meaningless.

Thus, among the different possibilities of fluid (aqueous and/or organic) uptake are the individual alternatives of: absorption; adsorption; cohesion; adhesion; covalent bonding; non-covalent bonding; hydrogen bonding; miscible envelopment; molecule entrapment; solution-uptake between fibers; fiber wetting; as well as others well documented in the scientific literature. Any and/or all of these may contribute to organic and/or aqueous fluid uptake in whole or in part. Which mechanism of action among these is actively in effect in any instance or embodiment is irrelevant.

2. By choosing a particular chemical formulation and/or desired stereoscopic (or three-dimensional) structure for the synthetic substance of the fabrication, the resulting biologically active textile can be prepared as a fabric having a markedly long functional duration and lifespan for in-vivo use. Accordingly, by choosing one or more durable and highly resilient chemical compositions as the fibers of choice, textiles effective for many years' duration and utility may be routinely made. All of these choices and alternatives are conventionally known and commonly used today by practitioners in this field.

It is also well recognized that some synthetic chemical compositions are available in a range of diverse formulations. As one example of a highly resistant chemical composition having many alternative formulations are the polyethylene terephthalates, of which one particular formulation is sold under the trademark DACRON.

As is commonly known in this field, a range of differently formulated polyethylene terephthalates (or “PETs”) are known to exist and are commercially available, each of these alternatives having a different intrinsic viscosity [or “IV”, as measured in o-chlorophenol or “OCP”, at 25° C.]. Typically, these differently formulated polyethylene terephthalate compounds can vary from less than 0.6 dl/g [IV] to greater than 1 dl/g [IV]; yet each of these alternative polyethylene terephthalate formulations can be dissolved in ice-cold 100% hexafluoroisopropanol. Thus, the electrospinning of appropriately prepared HFIP solutions containing any of such alternatively formulated polyethylene terephthalates will result in the fabrication of nanofibrous textile fabrics which are capable of independent or combined release of many diverse drugs, proteins and genetic materials.

3. The fibers comprising the agent-releasing textile (and the subsequently generated medical article or device) can be prepared in a variety of organizations as a tangible structure. Thus, as conventionally recognized within the textile industry, the textile fabric may vary in size or thickness; and may optionally receive one or more interior and/or exterior surface treatments to enhance particular attributes such as increased in-vivo biocompatibility or a greater expected time for functional operation and use in-vivo. All of these organizational variances are deemed to be routine matters which will be optionally chosen and desirably used to meet particular medical needs or individual patient requirements.

4. The fibers comprising the agent-releasing textile (and the subsequently generated medical articles or devices) can be prepared to meet the particulars of the intended in-vivo medical use circumstances or the contingencies of the envisioned clinical/therapeutic application. Thus, the textile fabric can alternatively be prepared either as a relatively thin-walled biocomposite, or alternatively as a thick-walled material; be produced as an elongated object having a diverse range of different outer diameter and inner diameter sizes; and be fashioned as a relatively inflexible or unyielding item or as a very flexible and easily contorted length of matter.

### B. The Choosing Of An Appropriate Biologically Active Agent

A number of different biologically active agents can be beneficially and advantageously utilized in tandem with the nanofibrous textile fabric. However, there are several minimal requirements and qualifications which the biologically active molecule—whatever its particular composition and formulation as a chemical compound, composition or molecule—must demonstrably provide in order to be suitable for use in the present invention. These are:
(i) The chosen agent must be capable of demonstrating its characteristic biological activity before becoming temporarily bound to and immobilized by the material substance of the fabricated textile. This characteristic biological activity must be well recognized and will constitute its ability/capacity to function as an active mediator in-situ.
(ii) The particular agent immobilized upon or within the material substance of the textile fabric must be capable of demonstrating its characteristic biological activity (its mediating capacity) after becoming immobilized and bound; and
(iii) The immobilized agent bound into the material substance of the textile fabric will be released in-situ and be delivered into the surrounding local environment as a freely mobile molecule which retains its characteristic biological activity (its mediating capacity) over an extended period of time after the agent-releasing textile has been utilized in-vivo and allowed to take up water.

In addition, since the primary medical application for the fabricated textile is expected to differ and vary extensively from one embodiment to another, it is intended that the characteristic biological properties of the chosen agent serve to aid, promote, and/or protect the naturally occurring pathways and processes of the body which occur in-vivo.

Accordingly, it is deemed likely that the primary function and capabilities of the chosen biologically active molecule will differ and vary in many instances; and thus there are multiple purposes and a range of individual goals for the releasable substance, among which are the following: (1) to serve as an antimicrobial agent—i.e., as an anti-bacterial or anti-fungal composition having a broad or narrow spectrum of activity; (2) to function as an anti-neoplastic compound effective against specific kinds of tumors; (3) to operate as a selective physiological aid—i.e., as a mediator which serves to avoid vascular complications such as blood coagulation or acts to prevent the formation of blood clots; and (4) to act as a pharmacological composition—i.e., as a drug or pharmaceutical which deactivates specific types of cells and/or functions to suppress or inhibit a variety of different humoral and cellular responses associated with or related to inflammation and the inflammatory response in-vivo. Examples of each are presented hereinafter.

### 1. The Releasable Antimicrobials of Choice

The Fluoroquinolone Antibiotics

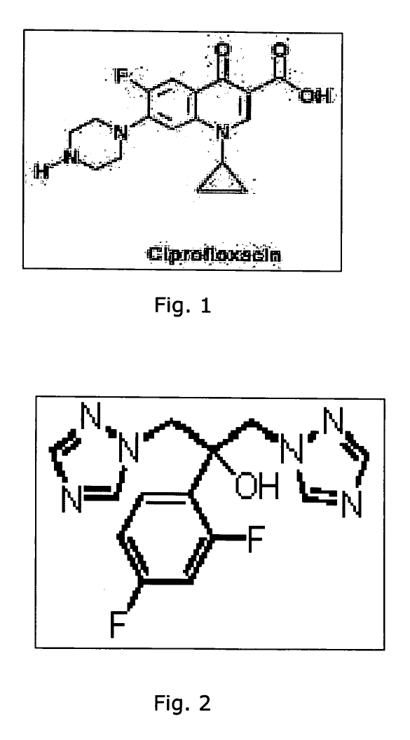

Antibiotics vary in structural type, spectrum of activity, and clinical usefulness. Fluoroquinolones such as Ciprofloxacin (hereinafter “Cipro”) are shown structurally by FIG. 1, and are of particular use and value in this invention. Quinolone antibiotics are chemically stable, and effective at low concentrations against the common clinically encountered organisms, particularly those bacteria responsible for biomaterial infection. These antibiotics also have structural features (solubility, molecular mass, and functional groups) that coincide with those of textile dyes known to have interactions with polyethylene terephthalates.

This family of antibiotics now includes at least twelve members—Ciprofloxacin, Ofloxacin, Norfloxacin, Sparfloxacin, Tomafloxacin, Enofloxacin, Lovafloxacin, Lomefloxacin, Pefloxacin, Fleroxacin, Avefloxin, and DU6859a; and the fluoroquinolone family as a whole has become the drug of choice for many applications. These antibiotics are effective at low concentrations; and hold an ideal antimicrobial spectrum against microorganisms most commonly encountered clinically in wound infection, with significant activity against many relevant pathogens—such as S. aureus, methicillin-resistant S. aureus, S. epidermidis, Pseudomonas species, and Escherichia coli. Moreover, Fluoroquinolones are heat stable; are of 300-400 r.m.m.; and have many structural features analogous to dyes. Accordingly, this family of antibiotics possesses those characteristics which are highly desired for use with the present invention.

A list of some representative antimicrobial/antiseptic agents that can be used solely or in conjunction with the fluoroquinolones is given by Table 3 below.

TABLE 3
Representative Antimicrobial/Antiseptic Agents

β-lactams
Biguanides
Cephalosporins
Chloamphenicol
Macrolides
Aminoglycosides
Quaternary Ammonium Salts
Tetracyclines
Sulfur-containing antimicrobials
Silver-containing compounds
Bis-Phenols (Triclosan)
Vancomycin
Novobiocin
Steriods (Fusidic acid)

The Anti-Fungal Agents

Development of antifungal agents has been on the rise over the past two decades due to a significant increase of superficial (i.e. nail beds) and invasive (i.e. blood-borne and medical-device related) infections. Fluconazole, known as Diflucan, a triazole-structured antifungal agent introduced in early 1990 and structurally shown by FIG. 2, has emerged as one of the primary treatments for Candida infections. The mode of action of Diflucan is the inhibition of 14α-lanosterol demethylase in the ergosterol biosynthetic pathway, and results in the accumulation of lanosterol and toxic 14α-methylated sterols in the fungal membrane. Similar to the selection of Cipro, Diflucan has structural features (solubility, molecular mass, and functional groups) that coincide with those of textile dyes known to have interactions with polyethylene terephthalate fibers. A agent-releasing textile combining polyethylene terephthalate with a slow-releasing antifungal agent such as Diflucan will have a marked impact on topical and implantable biomaterials such as medicated pads (useful for nail bed and skin infections), tampons (using localized release for yeast infection) and catheter cuffs.

Other examples of anti-fungal agents typically will include those listed by Table 4 below.

TABLE 4
Exemplary Anti-Fungal Agents

Amphotericin B
Nystatin
Terbinafine
Voriconazole
Echinocandin B
Itraconazole

The Antimicrobial Peptides

A novel class of antimicrobial agents known as antimicrobial peptides (or “AMPs”) has been discovered during the past two decades. These “natural” antimicrobial agents, which consist of a large number of low molecular weight compounds, have been discovered in plants, insects, fish and mammals, including humans [see for example, Marshall SH & Arenas G.,. “Antimicrobial peptides: A natural alternative to chemical antibiotics and a potential for applied biotechnology”, J Biotech 6(2): 1(2003)]. These peptides, whose composition can range from 6-50 amino acids, have been shown to have an important role in innate immunity. There are 5 general classifications for AMPs [see for example, Sarmafilk A., “Antimicrobial peptides: A potential therapeutic alternative for the treatment of fish diseases”, Turk J Biol 26:201(2002)], which are based on the three-dimensional structure of the peptide as well as the biochemical characteristics. These groups consist of: (1) linear peptides without cysteine residues or hinge region; (2) linear peptides without cysteine residues and a high proportion of certain amino acids; (3) antimicrobial peptides with one disulfite bonds that form a loop structure; (4) antimicrobial peptides with two or more disulfite bonds; and (5) antimicrobial peptides that have been derived from other larger proteins via post-translational processing.

AMPs have shown broad spectrum antimicrobial activity against both gram-positive (i.e., Staphylococcus aureus and epidermidis) and negative (i.e., Pseudomonas aeruginosa, E coli) bacteria. Some AMPs have also been shown to be effective against fungus [see for example, De Lucca A. J., “Antifungal peptides: Potential candidates for the treatment of fungal infections”, Expert Op Invest Drugs 9(2):273 (2000); and Selitrennikoff CP, “Antifungal proteins”, Appl Environ Microbiol 67(7):2883 (2001) and several antibiotic-resistant bacteria such as Mycobacterium tuberculosis [see for example, Linde C M A, Hofffier S E, Refai E, Andersson M., “In vitro activity of PR-39, a proline-arginine-rich peptide, against susceptible and multi-drug resistant Mycobacterium tuberculosis”, J Antimicrob Chemother 47:575 (2001); Miyakawa Y, Ratnakar P, Rao A G, Costello M L, Mathieu-Costello O, Lehrer R I, Catanzaro, A., “In vitro activity of the antimicrobial peptides human and rabbit defensins and porcine leukocyte protegrin against Mycobacterium tuberculosis”, Infect Immun 64(3):926 (1996); and Sharma S, Verma I, Khuller G K, “Therapeutic potential of human neutrophil peptide 1 against experimental tuberculosis”, Antimicrob Agents Chemother 45(2):639 (2001)].

Although the mode of action by these peptides has not been fully elucidated, it is postulated that many of these peptides interact directly with the bacteria wall, creating small channels (pores) which causes membrane destabilization, thereby depleting the bacteria of its cytoplasmic content [see for example, Matsuzaki K., “Why and how peptide-lipid interaction utilized for self defense? Magainins and tachyplesins as archetypes”, Biochemica Biophys Acta 1462(1-2):456 (1999)]. While effective against bacteria walls, there appears to be limited affinity for eukaryotic cells possibly due to the different composition and net charge of the membranes. Several AMPs (i.e., Nisin and Daptomycin) have been recently approved by the FDA for commercial and medical markets. This acceptance paves the way for utilizing other AMPs such as pleurocidin. Additionally, federal standard testing procedures, which were used to provide safety and efficacy data for these AMPs, have been established. Other representative types of AMPs are presented by Table 5 below.

TABLE 5

Cationic peptides
Cecropins
Defensins
Thionins
Amino Acid-Enriched
Histone-Derived
Beta-Hairpin
Other Natural and Functional Proteins
Anionic Peptides
Asparitc Acid-Rich
Aromatic Dipeptides
Oxygen-Binding Proteins

The Analgesic Agents

Analgesic agents are widely used in human and veterinary medicine in order to prevent inflammation, thereby reducing pain and other symptoms such as itching and swelling. These agents have structural properties that are comparable to standard textile dyes such as molecular weight, functional groups and benzene-ring based composition.

Exemplifying such analgesic agents are those listed by Table 6 below.

TABLE 6
Analgesic Agents:

Diphenhydramine Hydrochloride
Hydrocortisone Acetate
Pramoxine Hydrochloride
Lidocaine
Benzocaine

The Anti-Viral Agents

Antiviral agents have been used to combat viral infections ranging from the flu to HIV infection and organ transplant rejection.

Examples of some antiviral agents are given by Table 7 below.

TABLE 7
Antiviral agents

Oseltamivir (Flu)
Zanamivir (Flu)
Saquinavir (HIV)
Ritonavir (HIV)
Interferon (HIV/Implant Rejection)

### 2. The Releasable Anti-Neoplastic Agents

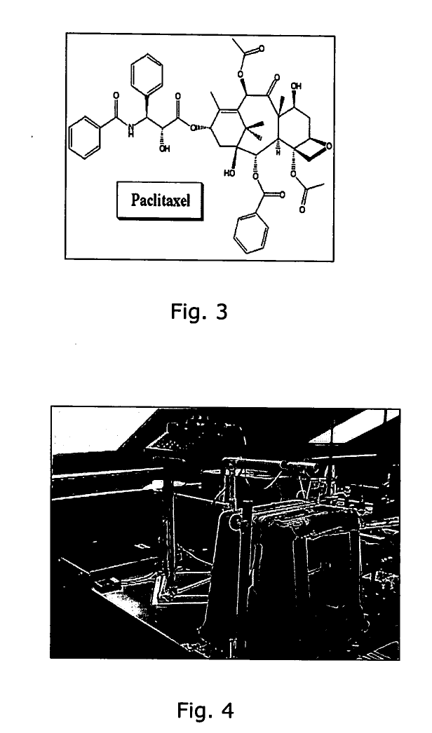

Paclitaxel, also known as Taxol, a diterpenoid-structured molecule shown by FIG. 3, is a potent anti-neoplastic agent. Paclitaxel has been shown to inhibit vascular smooth muscle cell (VSMC) proliferation, migration and inflammation. Additionally, Paclitaxel has been shown to inhibit the secretion of extracellular matrix by VSMCs, a major component of neointima formation leading to vessel restenosis. Paclitaxel stabilizes and enhances assembly of polymerized microtubules, an important component of the cytoskeleton involved in cell division, cell motility and cell shape.

Additionally, microtubules are involved in signal transduction, intracellular transport and gene activation. Paclitaxel has shown promise as a treatment for various types of cancers as well as for the prevention of restenosis following stent placement.

Nevertheless, when Paclitaxel is incorporated into a hydrophobic carrier polymer coated onto a metallic stent, it elutes for only 10-14 days. Other research groups have attempted to incorporate Paclitaxel into biodegradable polymers that would comprise the stent. However, Paclitaxel activity was significantly reduced due to the melt extrusion process for the fibers.

This issue would not be a problem with the present invention due to the low temperature formation of the nanofibrous polyethylene terephthalate (PET) fibers. Therefore, the fabrication of a nanofibrous polyethylene terephthalate (PET) material with a slow-releasing anti-neoplastic agent such as Paclitaxel would be particularly effective and medically applicable to endovascular stents and prosthetic vascular grafts, both of which currently experience neointimal hyperplasia.

Additional examples of other active anti-neoplastic agents suitable for use in the present invention include those listed by Table 8 below.

TABLE 8
Other Anti-Neoplastic Agents

Rapamycin
Dexamethasone

### 3. Other Classes Of Suitable Biologically Active Agents

A number of other classes of biologically active agents can also be used in the agent releasable textile. All of these choices are biochemical mediators which can be initially immobilized via the electrospinning technique without serious deterioration, and then subsequently released from the nanofibrous textile fabric upon uptake of water. Representative examples of such classes comprising additional suitable biologically active agents are presented by Tables 9, 10, and 11 respectively below:

TABLE 9
Releasable Proteins and Proteinaceous Matter

Growth Factors
Platelet derived growth factor (PDGF);
Epidermal growth factor (EGF), also known as vascular endothelial
growth factor (VEGF);
Macrophage derived growth factor (MDGF);
Fibroblast growth factor (FGF); and
Nerve growth factor (NGF).
Blood Anti-Coagulation Proteins
Antibodies or peptide fractions specific for any of blood Factors I-XII
respectively;
Antibodies or peptide fractions specific against Vitamin K;
Hirudin; and
Albumin.
Selected Cytokines (Enzymes)
Interleukin-1 (IL-1), an endogenous pyrogen and major inflammatory
mediator;
Interleukin-2 (IL-2), a T-cell activator and growth factor;
Interleukin-3 (IL-3), a hematopoietic growth factor;
Interleukin-4 (IL-4), a T-cell and B-cell growth factor;
Interleukin-5 (IL-5), a promoter of eosinophil growth and
differentiation and IgA antibody synthesis;
Interleukin-6 (IL-6), a B-cell differentiation factor;
Interleukin-7 (IL-7), a growth factor for early B- and T-
lymphocytes;
Interleukin-8 (IL-8), a chemotactic factor for neutrophils and
lymphocytes;
Interleukin-10 (IL-10), a down-regulator of cell activation;
Interleukin-12 (IL-12), an augmenter of IFN-γ production;
Interleukin-13 (IL-13), a factor which overlaps in function with IL-4;
Tumor necrotic factor (TNF), a factor which overlaps in function with
IL-1 and mediates host response to gram-negative bacteria;
Interferons-α -β, -γ, which activate macrophages, enhance
lymphocyte and natural killer cells, and have antiviral and antitumor
activity; and
Granulocyte-macrophage colony stimulating factor (GM-CSF), a growth
factor for granulocytes, macrophages, and eosinophils
Lectins (Mitogenic Agents From Plants)
Concanavalin A, a protein from the jack bean;
UEA I.
Glycoproteins And Proteoglycans
Ovalalbumin;
Avidin.

TABLE 10
Genetic Materials
Oligonucleotides

SiRNA
RGD (a protein/peptide coding sequence);
VCAM;
ICAM;
PCAM.

TABLE 11
Other Releasable Active Compositions
Saccharides And Polysaccharides

Glucosamine;
Chondroitin and chondroitin 4-sulfate;
Hyaluronic acid;
Heparin.

### III. The Unique Electrospinning Perfusion Method Of Manufacture

### A. The Steps Comprising The Electrospinning Perfusion Technique

1. The Generation of Nanofibrous Tubular Structures

A preferred method for making the agent-releasing textile of the present invention is via the unique technique of electrospinning perfusion. For this purpose, an electrospinning perfusion assembly is erected which comprises, at a minimum, a rotating mandrel which can be set at a pre-selected rotation speed; a needle fronted perfusion instrument, such as a syringe, which can be set to deliver a liquid mixture at a pre-specified flow rate; an electrical coupling for controlling and coordinating the electrical voltage applied across the perfusion needle and which is grounded to the rotating mandrel; and a controllable supply of electrical power. Utilization of this assembly permits uniform coating of the liquid mixture onto the surface of the mandrel; and the applied electrical voltage can be varied as needed to control the formation of the nanofibers upon the mandrel's surface.

Also for use in this erected assembly, a prepared mixture of chosen synthetic material and the biologically active agent of choice is blended together into an organic liquid carrier. For example, one preferred liquid mixture or blending is obtained by combining 20% w:v polyethylene terephthalate (PET) with 1.5% w:v of an antimicrobial (e.g., Cipro or Diflucan), or with 1.5% w:v of an anti-neoplastic compound (e.g., Paclitaxel), in a sufficient quantity of ice-cold hexafluoroisopropanol (hereinafter “HFIP”). A 10 ml syringe with a stainless steel 18-gauge blunt spinneret (0.5 mm internal diameter) is then filled with the liquid polymer blending and placed onto the Harvard Apparatus syringe pump.

It will be recognized in particular that electrospinning over a broad range of conditions is possible for polyesters. Thus, a range of differently formulated polyethylene terephthalates (or “PETs”) of intrinsic viscosity [or “IV” as measured in OCP at 25° C.] that range from less than 0.6 dl/g [IV] to greater than 1 dl/g [IV] can be dissolved in ice-cold 100% hexafluoroisopropanol. Electrospinning appropriately prepared HFIP solutions of such polyethylene terephthalates results in the fabrication of nanofibrous textile fabrics capable of independent or combined release of diverse drugs, proteins and genetic materials.

A Small Batch System

For fabricating small batches of product using this unique method, a chemically resistant syringe with a stainless steel blunt spinneret can serve as a functional instrument for perfusion. Alternatively, of course, any other tool, assembly or instrument capable of performing perfusion at a pre-selected flow rate and low reaction temperature can be usefully employed.

In this small batch system, the perfusion syringe of the assembly is filled with the prepared liquid mixture described above and placed onto a Harvard Apparatus syringe pump. The perfusion rate is preferably set at 3 ml/hour at 25° C. If desired, however, the flow rate can be increased and/or decreased to meet specific requirements. Similarly, the reaction temperature is preferably ambient room temperature (20-25° C.), but when necessary or desired can be chosen to be within a temperature reaction range of about 0-50° C.

A PTFE-coated stainless steel mandrel (diameter=4 mm) is preferably set at a jet gap distance of 15 cm from the tip of the syringe needle. Gap distance can be varied at will to change the fiber diameter size. The rotable mandrel was then electrically grounded to the power source, with the positive high potential source connected to the syringe needle. The mandrel rotates or spins at a pre-selected rate of rotation throughout the act of liquid perfusion.

Perfusion

Perfusion of the polymer solution begins upon application of the electric current to the tip of the syringe needle (typically 15 kV), which then moves at a preset constant speed and fixed distance from the mandrel surface for a limited time period (typically about 40-60 minutes in duration). This process of manufacture is therefore termed “electrospinning perfusion”; and yields a fully fabricated, elongated nanofibrous textile conduit whose inner diameter size corresponds to the overall diameter of the mandrel (in this instance, 4 mm).

When using a single nozzle (or syringe needle), it was that increasing electrospinning time significantly beyond about 40 minutes increased the rigidity of the resulting nPET material. However, multiple nozzles (or syringe needles) can be used concurrently to reduce the time required to fabricate tubular structures of the appropriate rigidity. The use of multiple injection streams to increase production rates is a familiar concept to those skilled in the art; and, accordingly, the use of multiple nozzles lies within the scope of the present invention.

Optional Follow-Up Processing

When the process is used to make certain kinds of medical articles such as synthetic vascular graft prostheses, a crimping procedure is employed as an optional, but very desirable, follow-up process. Accordingly, after being formed as a hollow tube by electrospinning perfusion, the thickness and girth of the originally formed fibrous composite wall and exterior surface preferably is then intentionally altered into a crimped structural form via a limited heat set technique, followed by compression of the fibrous composite wall, in order to provide kink-resistance for the elongated tube.

In brief, the end portions of the formed hollow tube (appearing about 1 cm from each end of the mandrel) are cut off and discarded. The remainder of the elongated hollow tube is then stretched 25% of the starting segment size while on the mandrel in order to provide a set strain across the fibers, a manipulation that occurs in normal fiber extrusion. The stretched tubes are then immediately exposed to 100% ethanol for 2 hours time at room temperature (or in 100% ethanol for 30 minutes with sonication) in order to remove the residual solvent, followed by air-drying overnight at room temperature.

2. The Generation of Flat Sheet Nanofibrous Textile Fabrics

Similar in its essentials to the technique described above, DACRON chips were dissolved in ice-cold 100% hexafluoroisopropanol (19% w:v) and mixed on an inversion mixer for 48 hours in order completely solubilize the chips. The self-contained, semi-automated electrospinning apparatus containing a Glassman power supply, a Harvard Apparatus syringe pump, an elevated holding rack, a modified polyethylene chamber, a spray head with power attachment and a reciprocating system was again used.

The Wheaton stirrer was used to provide a holding chamber for the new flat collecting plate employed to generate a sheet format. The design of this surface is based upon the collecting plate employed by Li et. al. [see Li W J, Laurencin C T, Caterson E J, Tuan R S, Ko F K., “Electrospun nanofibrous structure: A novel scaffold for tissue engineering”, J Biomed Mater Res 60:613 (2002)]. In short, a flat 12 cm ×10 cm copper plate, containing a 6 cm stainless steel rod extending from the underside of the plate was designed and grounded to the power source.

A 10 ml chemical-resistant syringe was filled with the polymer liquid. A stainless steel 18-gauge blunt spinneret (0.5 mm internal diameter) was then cut in half, with the syringe fitting end connected to the polymer-filled syringe. Nalgene PVC tubing was connected to the syringe filled with the polymer solution followed by connection to the other half of the blunt spinneret within the spray head. The line was then purged of air, with the syringe then placed onto the syringe pump. The high potential source was connected to the spray head tip, with the plate set at a jet gap distance of 15 cm from the tip of the needle. The perfusion rate was set at 3 ml/hour at 25° C.

Perfusion of the polymer liquid was started upon application of the current to the tip of the needle (15 kV) with electrospinning proceeding for 1 hour and 40 minutes, with rotation of the plate 20° every 20 minutes. This resulted in a flat, planar sheet of nanofibrous textile material being formed.

### B. The Agent-Releasing Textile Fabricated By Electrospinning Perfusion Methods

The agent releasable nanofibrous textile formed by the electrospinning method described above has a number of unique structural features which are the direct result and characteristic of its unique mode and manner of manufacture.

1. The agent-releasing textile fabricated via one of the two different electrospinning perfusion techniques will yield a discrete tubular article of fixed inner-wall and outer wall diameters, and a solid wall girth and configuration formed of a nanofibrous composite composition. The material substance of the fabricated wall typically shows that the synthetic substance is present as discrete fibers about 10⁻⁸ meters in diameter size. The fiber size is clearly demonstrated by the empirical data presented subsequently herein.

2. The interior wall surface and the exterior wall surface of the tubular structure comprising the agent-releasing textile are markedly different owing to the crimping and heat setting treatments following the initial electrospinning perfusion steps of the methodology. Thus, the exterior wall surface can possess a crimped and a somewhat irregular appearance. In comparison, the interior wall surface and the internal lumen of the conduit as a whole presents a smooth, regular, and even appearance which is devoid of perceptible projections, lumps, indentations, and, roughness.

3. The nanofibrous composite material substance of the textile fabric, whether existing in tubular structure form or in planar sheet form, is resilient and can be prepared in advance to provide varying degrees of flexibility, springiness, suppleness, and elasticity. Moreover, the nanofibrous biocomposite wall is durable and strong; is hard to tear, cut, or breakup; and is hard-wearing and serviceable for many years' duration.

4. The nanofibrous material substance of the agent releasable textile, whether present in tubular structure form or in planar sheet form, is biocompatible with the cells, tissues and organs of a living subject; and can be implanted surgically in-vivo without initiating or inducing a major immune response by the living host recipient. While aseptic surgical technique and proper care against casual infection during and after surgery must be exercised, the agent releasable textile can be usefully employed for a variety of applications in-vivo.

### C. The Major Benefits And Advantages Of The Electrospinning Perfusion Techniques

The electrospinning perfusion technique—whether employed to fabricate tubular structures or flat sheets, has a number of advantages over conventionally known manufacturing processes. These include the following:

A first benefit is that no exogenous binders, cross-linking compounds, or functional agents are required by the process either to form the substance of the fabric or to maintain the integrity of the fabricated textile. The synthetic substance prepared in liquid organic solvent can be generated directly into nanofibrous fabric form via the low reaction temperatures (typically ranging between 0-50° C.) permitted and used by the electrospinning perfusion process. In addition, the nanofibers of the fabric act to seal the interstices of the composite material; therefore, no sealants as such are required. This manufacturing technique also benefits the manufacturer in that the technology is not a dipping or immersion method of preparation, which can be awkward and difficult to perform; or is a process which typically requires the addition of heat, such as if a conventional melt spinning method of fiber formation were employed.

A second benefit is that the electrospinning perfusion technique yields a textile fabric formed as a nanofibrous composite in which the fibers (e.g., PET) exist independently and are visibly evident throughout the material of the textile. This structural distribution of discrete fibers within the fabric adds strength and flexibility to the textile as a whole. Also, the presence of these fibers collectively provides sites into which diverse biological agents (such as antimicrobials, anti-neoplastic agents, and the like) can be temporarily incorporated and indefinitely, although non-permanently, immobilized until such time as the textile takes up fluid—i.e., any aqueous and/or organic liquid.

A third benefit is the capability for direct incorporation of biologically-active agents onto the nanofibrous material, whatever its final shape and structure. This process holds several key advantages over other conventionally known methodologies in that:
The active agent is incorporated into the fabricated nanofibrous material without molecular modification, and is non-permanently immobilized within each individual fiber surface as the individual fibers are formed.
No one particular mechanism of incorporation is responsible for the active agent becoming non-permanently immobilized within each individual fiber of the fabricated nanofibrous material; and thus any and all of the commonly known mechanisms—such as absorption, adsorption, polarity, ion attraction, and the like—may be involved.
The amount of active agent can be adjusted within the bulk polymer depending on the specific or intended application.
No cross linking agents are needed, or used, or desired at all, thereby avoiding concerns over drug carrier toxicity, biocompatibility, and mutagenicity.
Low reaction temperatures are used during the fiber/fabric formation procedure, thus maintaining the biologic activity of the active agent.
Active agent elution from the textile fabric is controlled and sustained over time, as shown in the experimental studies and empirical data presented hereinafter.

### IV. The Medical Articles Fashioned From The Agent Releasable Textile

It is expected and envisioned that each agent-releasing textile can be employed in the alternative either (1) as a configured tubular conduit whose internal lumen is usefully employed for the conveyance of fluids in-situ; or (2) as a solid mass of flat or planar nanofibrous sheet fabric which achieves its intended purpose without regard to or actual use of any internal lumen within the textile fabric. Some representative examples of the former format are given by the listing of Table 12 and illustrative examples of the latter format are provided by the listing of Table 13 below.

TABLE 12
Embodiments Using The Tubular Structure Format

Vascular articles
Arterial vascular grafts;
Venous vascular grafts;
Prostheses for aneurysms;
Liners and covers for stents (coronary or endovascular).
Non-vascular devices
Catheter cuffs
Coating for wires for transdermal devices (pacemaker leads)

TABLE 13
Embodiments Using the Flat Sheet Format

Wound dressings
treatment dressings, films, and/or sheets;
gauze pads;
absorbent sponges;
bandages; and
sewing cuffs.
Trans-dermal release patches
Infection treatment;
Skin tumor treatments; and
Finger/toenail treatment
Personal hygiene products
Tampons; and
Contraceptive delivery

### V. Some Intended Clinical/Therapeutic Applications For The Invention

The kinds of clinical/therapeutic applications for the prepared medical articles and devices are intended to include major traumatic wounds caused by accident, negligence, or battlefield conditions; planned surgical incisions and invasive body surgical procedures performed under aseptic conditions; transcutaneous incisions and vascular openings for catheter insertion and blood vessel catheterization procedures; and other body penetrations and openings made for therapeutic and/or prophylactic purposes.

The medical articles provided by the present invention thus are intended and expected to be manufactured as pre-packaged and pre-sterilized textile fabric articles; be an item which can be prepared in advance, be stocked in multiples, and be stored indefinitely in a dry state without meaningful loss of biological function or efficacy; and serve effectively in the treatment of disease, disorders, and pathological conditions under many different clinical circumstances.

The medical articles should be manufactured and tailored in advance to meet a wide range of intended use circumstances or contingencies expected to be encountered in a particular situation. For this reason, the constructed textile article can and should alternatively be prepared as a thick cloth and as a thin gauze; as a solid-walled configured tube; and as a delicate film. Equally important, the resulting construct may take physical form either as a stiff, inflexible and unyielding mass or as a very flexible and supple layer; have a varied set of dimensions and girth; appear as both a geometrically symmetrical or asymmetrical configured fabric; and can exist even as a slender cord or string-like length of material.

Medically, the agent releasable textile articles of the present invention can be employed in-vivo in the following ways: topically or subtopically; transcutaneously, percutaneously, or subcutaneously; or internally within the body's interior; vascularly or humorally; and applied to any kind of body cavity, body tissue or body organ without regard to anatomic site or location.

### VI. Experiments, Empirical Data, and Results

To demonstrate the merits and value of the present invention, a series of planned experiments and empirical data are presented below. It will be expressly understood, however, that the experiments described herein and the results provided below are merely the best evidence of the subject matter as a whole which is the present invention; and that the empirical data, while limited in content, is only illustrative of the scope of the present invention as envisioned and claimed.

An illustrative recitation and representative example of the present invention is the preferred manner and mode for practicing the methodology is also presented below as part of the experimental method. It will be expressly understood, however, that the recited steps and manipulations presented below are subject to major variances and marked changes in the procedural details; all of which are deemed to be routine and conventional in this field and may be altered at will to accommodate the needs or conveniences of the practitioner.

### Series A: Preparation and Characterization Of Nanofibrous (nPET) Textiles

Experiment 1. The Electrospinning Perfusion Technique

The Electrospinning Apparatus

For small batch purposes, a self-contained semi-automated electrospinning perfusion apparatus was assembled which included a Glassman power supply, a Harvard Apparatus syringe pump, an elevated holding rack, a modified polyethylene chamber, a spray head with power attachment, a reciprocating system, and a Wheaton stirrer for controlled mandrel rotation. Such an assembly is shown by FIG. 4.

Utilization of this assembly permits uniform coating of a liquid polymer onto the PTFE-coated stainless steel mandrel (diameter=4 mm). A 10 ml chemical-resistant syringe was filled with the liquid polymer; and a stainless steel 18 gauge blunt spinneret (0.5 mm internal diameter) was cut in half, with the syringe fitting half connected to the chemical-resistant syringe.

Nalgene PVC tubing ( 1/32 ID× 3/32 OD; 66 cm length) was then connected to the syringe, followed by connection to the other half of the blunt spinneret within the spray head. The line was purged of air, with the syringe then placed onto the syringe pump. The high potential source was connected to the spray head tip; and the mandrel was set at a jet gap distance of 15 cm from the tip of the needle. The mandrel was then grounded to the power source; and the perfusion rate was set at 3 ml/hour at 25° C.

The Liquid Polymer Blend

A polyethylene terephthalate (20% w:v) polymer liquid was prepared in ice-cold 100% hexafluoroisopropanol. The 10 ml syringe with a stainless steel 18-gauge blunt spinneret (0.5 mm internal diameter) was filled with the liquid polymer blending and placed onto the Harvard Apparatus syringe pump.

The Perfusion Technique

Perfusion of the polymer was then started upon application of the current to the tip of the needle (15 kV) with electrospinning proceeding for 40 minutes. After electrospinning, the end portions of the resulting tubular structures comprised of nanofibrous polyethylene terephthalate, now termed “nPET” structures, were cut off and discarded (1 cm from each end of the mandrel). The original nPET tubular structures were then stretched 25% of the starting segment size while on the mandrel in order to provide a set stain across the fibers, a process that occurs in normal fiber extrusion. This yielded sized tubular segments of nPET fabric.

Some, but not all, of the stretched nPET segments were then immediately exposed to 100% ethanol for 2 hours at room temperature (or for 30 minutes in 100% ethanol with sonication) in order to remove the residual solvent. Then, all of the nPET tubular structures (ethanol exposed or not) were air-dried overnight at room temperature.

Results

The nPET tubular segments, whether air-dried or exposed to ethanol followed by air-drying, had a consistent 4 mm internal diameter throughout the lumen (length=7.5 cm). A total of 4 nPET structures were synthesized for each method using the above-described process.

For this experimental study, the nPET segments air-dried at 60° C. were employed for all of the subsequently conducted in-vitro studies reported herein. This post-synthesis treatment was performed owing to the possibility of Cipro eluting during the ethanol incubation for the other methodology described later herein.

Concerning the electrospinning technique itself for tubular structures fabricated using the described parameters, it was found that increasing electrospinning time significantly beyond 40 minutes increased the rigidity of the resulting nPET material. Conversely, electrospinning the liquid polymer blending for shorter periods of time (e.g., 1-15 minutes) provided a tubular structure without significant (less than 1 pound break strength) wall strength. Major differences in and variance of tubular wall rigidity may be desired for the various medical articles and devices to be employed clinically. However, the chosen parameters employed for nPET material formation in these experimental studies were uniformly and consistently maintained at 40 minutes of electrospinning time, a polymer concentration of 20%, an applied voltage (15 kV), and a gap distance of 15 cm.

### Experiment 2: Characterization Of Physical Properties Of Electrospun nPET Material

Tensile Strength/Ultimate Elongation

Tensile strength (pounds force), strain at maximum load (%) and strain at break (%) for knitted DACRON segments (formed of a commercially obtained standard textile material) and for electrospun nPET segments (formed of a polyethylene terephthalate compound prepared as described above) were measured using previously published techniques. Control and test segments (7 mm width, 3 cm length; n=3/test condition) of both kinds of material were measured and cut.

A Q-Test Tensile Strength Apparatus (MTS Systems, Cary, N.C.) was calibrated according to manufacturer's specifications in a climate-controlled environment (room temperature =67° F., 45% relative humidity). Each of the samples under test were also conditioned in this environment for 24 hours. Segment stretching (crosshead speed=50 mm/min, gauge length=2 cm, load cell=25 lb) was then initiated and terminated upon segment breakage.

Results

There was a marked difference between the break load of knitted DACRON segments (42±9 pounds force) and electrospun nPET segments (3.7±0.9 pounds force). This difference in breaking load was expected owing to the significantly greater wall thickness of the knitted DACRON material. The other physical properties, such as the percent strain at maximum load (60±24 versus 55±8) and percent strain at break (60 versus 62±3), were comparable between the two test materials, indicating that the difference in break strength was directly related to wall thickness. Thus, the nPET material is shown to possess significant physical characteristics that would permit its presence and application in various medical devices.

### Experiment 3: Evaluation Of Electrospun nPET Material Via Scanning Electron Microscopy

Scanning Electron Microscopy (SEM)

Two electrospun nPET segments were randomly selected and examined via a JEOL JSM 5900 LV electron microscope in order to determine fiber size and distribution throughout the material wall.

Results

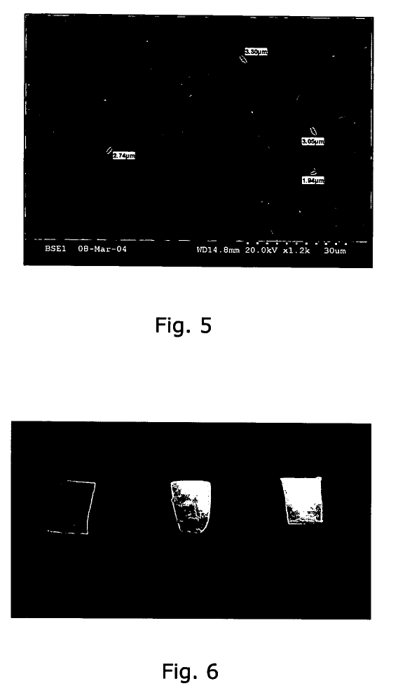

Analysis of electrospun nPET tubular structures via SEM revealed that the diameter of the polyethylene terephthalate fibers comprising the nanofibrous material varied from about 100 nm to 3000 nm in size. This is shown by the microphotograph of FIG. 5. A comparison SEM analysis of the knitted DACRON samples revealed that the knitted DACRON fibers ranged from 15 to 30 μm in diameter size (data not shown) and thus were significantly larger than the nPET fiber diameter size range.

### Series B: The Agent-Releasing Textiles Comprising The Present Invention

### Experiment 4: Synthesis Of Novel nPET Materials With Biologically Active Agents

Prior to forming the blended polymer solution, the solubility of Cipro, Diflucan and Paclitaxel in the HFIP (hexafluoroisopropanol) solvent was determined. Based on the pre-chosen concentration of active agent to be employed in the composite, 15 mg of each respective agent was placed into 1 ml of the HFIP solvent, mixed and observed.

Following this initial assessment, polyethylene terephthalate (19%) polymer solutions containing either Cipro, or Diflucan, or Paclitaxel (1.5% w:v) respectively were prepared.in ice-cold 100% hexafluoroisopropanol. These individually prepared polymer solutions of Cipro, or Diflucan, or Paclitaxel were mixed on an inversion mixer for 48 hours in order to completely solubilize both the polyethylene terephthalate polymer and each active agent component in their respective individual solutions. Then, the self-contained, semi-automated electrospinning apparatus (described previously herein) was again employed for fabricating each version of nanofibrous textile material.

Utilization of this system permits uniform coating of the prepared polyethylene terephthalate polymer solution onto the PTFE-coated stainless steel mandrel (diameter=4 mm). Using the uniform set of parameters of the previously described experimental series, the mandrel was set at a jet gap distance of 15 cm from the tip of the needle. The mandrel was then grounded to the power source. The perfusion rate was set at 3 ml/hour at 25° C. Perfusion of the polyethylene terephthalate/active agent mixture was then started upon application of the current to the tip of the needle (15 kV) with electrospinning proceeding for 40 minutes. After electrospinning, the end portions of the original tubular structure (1 cm from each end of the mandrel) were cut off and discarded. This resulted in textile tubular segments of fixed length.

The resulting tubular segments were then stretched 25% of the starting segment size while on the mandrel in order to provide a set strain across the fibers, a process that occurs in normal fiber extrusion. These tubular segments were then either air-dried at 60° C. overnight; or exposed to 100% ethanol for 2 hours at room temperature in order to remove the residual solvent. Due the fluorescent properties of Cipro, nPET segments (those having no active agent) and nPET-Cipro segments (those having Cipro as the active agent)—having been already exposed to 60° C. temperature overnight or to 100% ethanol for 2 hours—were then exposed to a hand-held UV light to qualitatively assess Cipro presence within the textile structure.

Results

Cipro, Diflucan and Paclitaxel individually were each found to have excellent solubility in the HFIP solvent. Once combined with the polyethylene terephthalate polymer/HFIP liquid, the solubility of each respective active agent remained unchanged. Formation of nPET (as a substantive material) and of nPET tubular structures containing either Cipro, or Diflucan, or Paclitaxel were all successfully accomplished. All these structures showed a consistent 4 mm internal diameter throughout the lumen for each tubular structure (material length=7.5 cm). Based on the perfusion rate in conjunction with electrospinning time, each tubular segment incorporated approximately 30 mg of each respective active agent.

In addition, similarly to our previous experimental series, increasing electrospinning time significantly increased the rigidity of the resulting nanofibrous material. Conversely, electrospinning for shorter periods of time (1-15 minutes) provided a tubular structure without significant wall strength.

Furthermore, gross observation of the various resulting tubular segments via UV illumination revealed intense fluorescence from the nPET-Cipro segments, whether air-dried or ethanol washed, when compared to the nPET segments. This UV illumination data demonstrated the presence of Cipro to be only within the nPET-Cipro segments. This effect is illustrated by FIG. 6.

### Experiment 5: Determination Of Cipro and Diflucan Release From nPET-Cipro And nPET-Diflucan Segments Via UV/VIS Spectrophotometer

Methods

nPET segments, nPET-Cipro segments, and nPET-Diflucan segments (0.5 cm segment length, n=3 segments/time interval/segment treatment) were individually placed into 5 ml of phosphate buffered saline (PBS) followed by continuous agitation using Rugged Rotator inversion mixer (33 r.p.m.) at 37° C. Wash solutions were sampled at acute (0, 1, 4 and 24 hours) and chronic (2-21 days for Cipro and 2-7 days for Diflucan) time periods, with replacement of the wash solution with a fresh 5 ml PBS after sampling. The absorbance of wash solutions were read at 322 nm (PBS blank) using a Beckman DU640 UV/VIS spectrophotometer.

A standard curve using known Cipro concentrations ranging from 0-100 μg/ml was prepared. This Cipro standard curve was then used to extrapolate the antibiotic concentration within the wash solutions.

Results

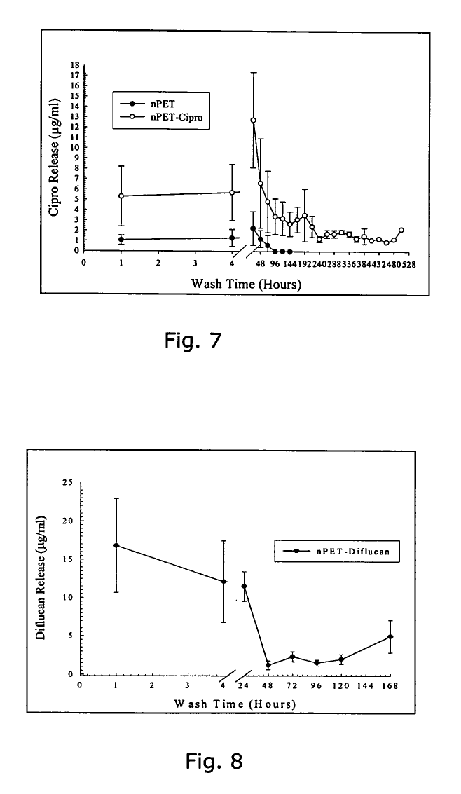

The release profiles for the nPET-Cipro segments are shown by FIG. 7, and the release profiles for the nPET-Diflucan segments are shown by FIG. 8. Notably, the release profiles for each type of segment are markedly different.

As observed and recorded, Cipro release within the first 4 hours was consistent at 5±2 μg/ml, and was followed by a sharp increase in rate to 13±4 μg/ml at 24 hours. Cipro release then decreased to 6±4 μg/ml by 48 hours, but persisted (ranging from 1-2 μg/ml) throughout the time duration of this study (504 hours). The amount of Cipro released has significant biological activity, owing to the low MIC₅₀ for Cipro (0.26 μg/ml).

In comparison, Diflucan release followed typical first order kinetics in that the greatest release occurred within the first 24 hours (17, 12 and 11 μg/ml, respectively). This was followed by a slow sustained release over the remaining time periods over the 168 hour study period, the time duration of this study.

Overall therefore, nPET segments containing Cipro and Diflucan demonstrated significant release of each active agent throughout the time periods empirically evaluated.

### Experiment 6: Antimicrobial Activity Of nPET Segments And nPET-Cipro Segments Via A Zone Of inhibition Assay

Methods

nPET segments (n=3 segments/time interval) and nPET-Cipro segments (n=9 segments/time interval), which were previously washed as described above, were then evaluated for antimicrobial activity using a zone of inhibition assay.

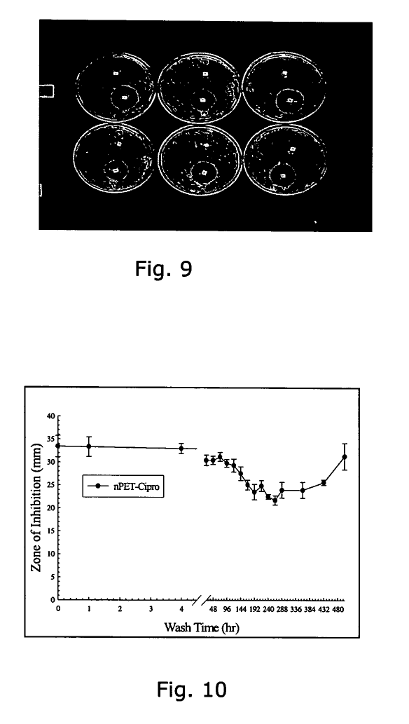

A stock solution of S. aureus was thawed at 37° C. for 1 hour. Upon thawing, 1 μl of this stock was added to 5 ml of Trypticase Soy Broth (TSB) and incubated overnight at 37° C. From this solution, 10 μl was streaked onto Trypticase Soy Agar (TSA) plates. nPET segments and nPET-Cipro segments were individually embedded into the S. aureus streaked TSA plates; and each prepared plate was then placed into a 37° C. incubator overnight. Standard 5 μg Cipro Sensi-Discs (n=3) were also embedded into the S. aureus streaked TSA plates at each time interval as a positive control. The zone of inhibition each piece was determined, taking the average of 3 individual diameter measurements. Zone size (mm) over time was determined for each parameter. The prepared assay plates are illustrated by FIG. 9.

Results:

The nPET-Cipro segments demonstrated significantly greater antimicrobial activity than nPET segment controls at all of time periods examined. This is graphically shown by the data of FIG. 10.

The zone of inhibition created by the 5 μg Cipro Sensi-Discs was consistent at 23 mm. The nPET-Cipro segment antimicrobial activity profile correlated with the Cipro release determined in the spectrophotometric studies—in that the greatest antimicrobial activity occurred within the first 48 hours. Cipro antimicrobial activity, presumably caused by lower Cipro concentrations being released over time as determined by the spectrophotometry, decreased slowly over the remaining time periods. Nevertheless, significant antimicrobial activity was still evident even after 504 hours, with inhibition zones being comparable to those of the Sensi-Disc results. Thus, this study demonstrates that Cipro release from the nPET material persisted for over 504 hours, with antimicrobial activity correlating to the quantity of Cipro release.

### Experiment 7: Anti-Fungal Activity Of nPET Segments And nPET-Diflucan Segments Using A Turbidity Assay

Methods

Candida albicans was purchased from ATCC. The fungus was re-hydrated in YM Broth with 0.5% dextrose and grown for 30 hours at 30° C. under humidified conditions. nPET segments and nPET-Diflucan segments (1 cm², n=2 segments/inoculum/treatment) were prepared as previously described herein, and then tested against various Candida albicans concentrations.

A broth macrodilution assay was performed based on the NCCLS M27-A protocol (Ref 62). The stock fungal inoculum concentration was determined via backplating a set volume of the diluted fungus broth onto Trypticase Soy Agar plates. The number of colony forming units (cfu) grown per plate was then counted and extrapolated to determine the starting Candida concentration.

The stock fungus solution was then diluted to 10⁶, 10⁵ and 10⁴ cfu/ml. After incubating the individual test segments in 2 ml of the fungus solutions for 24 hours at 30° C., the optical density of the broth solutions was measured at 492 nm (Ref 63). These values were compared to Candida solutions without any nPET materials (serving as the positive control) as well as against YM Broth only and Candida solutions with 40 μg Diflucan solution (both serving as negative controls).

Results

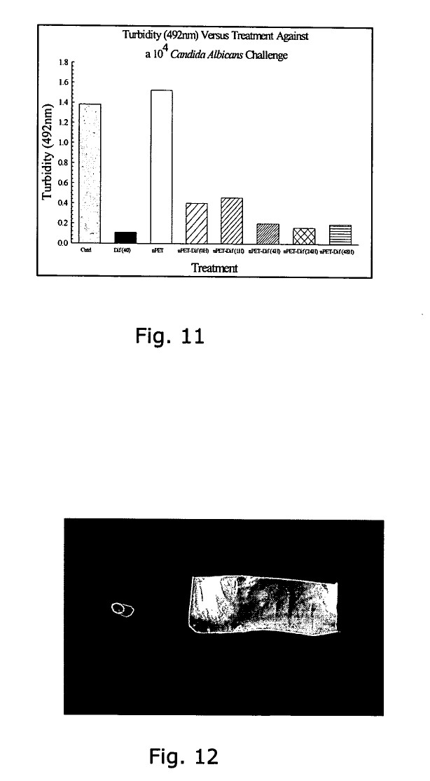

The nPET-Diflucan segments had significantly greater antifungal activity at all wash periods as compared to nPET segments which had no antifungal activity (turbidity comparable to Candida control). This is graphically shown by the data of FIG. 11.

Diflucan (40 μg) in solution demonstrated excellent antifungal activity against this inoculum, with decreasing activity as the inoculum increased. Antifungal activity by the nPET-Diflucan segments was clearly evident at all Candida concentrations evaluated with activity mimicking solution-based Diflucan (data not shown). Thus, this experimental study demonstrated that Diflucan is released from the electrospun nanofibrous material even after extensive washing for 2 days, with Diflucan maintaining it recognized and characteristic antifungal activity after synthesis of the nPET-Diflucan tubular structure.

### Experiment 8: Development Of Electrospinning Methodology For Flat Sheet Nanofibrous (nPET) Material

Methods

As described in Series A above, prepared polyethylene terephthalate chips were dissolved in ice-cold 100% hexafluoroisopropanol (19% w:v) and mixed on an inversion mixer for 48 hours in order completely solubilize the chips. The self-contained, semi-automated electrospinning apparatus containing a Glassman power supply, a Harvard Apparatus syringe pump, an elevated holding rack, a modified polyethylene chamber, a spray head with power attachment and a reciprocating system was again used.

The Wheaton stirrer was used to provide a holding chamber for the new flat collecting plate employed to generate a sheet format. The design of this surface is based upon the collecting plate. In short, a flat 12 cm×10 cm copper plate, containing a 6 cm stainless steel rod extending from the underside of the plate was designed and grounded to the power source.

A 10 ml chemical-resistant syringe was filled with the polymer liquid. A stainless steel 18-gauge blunt spinneret (0.5 mm internal diameter) was then cut in half, with the syringe fitting end connected to the polymer-filled syringe. Nalgene PVC tubing was connected to the syringe filled with the polymer solution followed by connection to the other half of the blunt spinneret within the spray head. The line was then purged of air, with the syringe then placed onto the syringe pump. The high potential source was connected to the spray head tip, with the plate set at a jet gap distance of 15 cm from the tip of the needle. The perfusion rate was set at 3 ml/hour at 25° C.

Perfusion of the polymer liquid was started upon application of the current to the tip of the needle (15 kV) with electrospinning proceeding for 1 hour and 40 minutes, with rotation of the plate 20° every 20 minutes. This resulted in a flat, planar sheet of nPET nanofibrous material being formed. The resulting nPET sheet is illustrated by FIG. 12.

After the electrospinning procedure was completed, a 1.0 cm margin around the perimeter edge of the entire nPET planar sheet was cut off in order to eliminate potential variability in the fabric thickness along the edge. The flat nPET sheet construct was then stretched 25% in the width and length of the material in order to provide a uniform set strain across the fibers, followed by air-drying at 60° C. overnight.

Results

A flat sheet of electrospun nPET textile fabric (8 cm×10 cm) was formed using this alternative method and technology. When viewed in gross, the nPET planar sheet had excellent handling characteristics and possessed physical properties comparable to the nPET tubular structures.

### VII. Conclusions Drawn From And Supported By The Empirical Data

1. The self-contained, semi-automated electrospinning apparatus provided by the present invention can be employed to generate two different formats of nanofibrous textile fabrics. One format is a tubular structure having determinable inner wall and outer wall diameter sizes, two open ends, and an internal lumen typically less than about 6 millimeters in diameter. This tubular structure format presents an interior wall surface and an exterior wall surface, and is a conduit biocompatible with and suitable for the conveyance of liquids and gases through its internal lumen.

A second format is a flat or planar sheet construction having determinable, length, width, and depth dimensions. The flat sheet fabric can be folded and refolded repeatedly; can be cut and sized to meet specific configurations; is resilient and can be prepared in advance to provide varying degrees of flexibility, springiness, suppleness, and elasticity.

2. A wide range and variety of agent-releasing textiles can be prepared for use as medical articles and devices using the present invention. The agents are biologically active and well characterized; are incorporated in chosen concentrations as an ingredient in the bulk polymer prior to making the textile fabric; and become indefinitely attached to and non-permanently immobilized upon the fabricated nanofibrous textile material as a concomitant part of the process for manufacturing the textile.

3. After being placed in a water containing environment, the agent-releasing textile will begin to take up water; release its incorporated biologically active agent in-situ over time; and deliver the release active agent at measurable concentrations directly into the adjacent and surrounding milieu. The in-situ released agent is function, operative and potent; and provides/performs its well recognized and characteristic biologically activity whenever and wherever it is delivered.

The present invention is not to be restricted in form nor limited in scope except by the claims appended hereto.

What we claim is:

1. A fabricated textile useful for the making of a medical article or device, said fabricated textile comprising:
a nanofibrous composite material comprised of at least one biodurable synthetic substance and fabricated as a flat sheet fabric via an electrospinning perfusion process, said flat sheet nanofibrous fabric having a determinable length, width, and depth, and being biocompatible with the tissues and organs of a living subject.

2. The fabricated textile recited in claim 1 wherein said biodurable synthetic substance is a polymeric composition.

3. The fabricated textile recited in claim 1 wherein said biodurable synthetic substance is a polymer selected from the group consisting of polyethylene terephthalate, nylon, polyurethane, polyglycolic acid, polyamides, polytetrafluoroethylene, polyesters, and mixtures of these substances.

4. The fabricated textile recited in claim 1 wherein said biodurable synthetic substance is a compound selected from the group consisting of an acetate, triacetate, acrylic, acrylonitile, aramid, modacrylic, olefin, propylene, ethylene, and saran.

5. An agent-releasing textile useful for the making of a medical article or device, said agent-releasing textile comprising:
a nanofibrous composite material comprised of at least one biodurable synthetic substance and fabricated as an elongated hollow tubular structure via an electrospinning perfusion process, said fabricated nanofibrous tubular structure having determinable inner and outer wall diameters, two open ends, and an internal lumen, and being biocompatible for the conveyance of fluid through its internal lumen; and
at least one pre-chosen biologically active agent having recognized and characteristic mediating properties which has been combined with said biodurable synthetic substance in liquid admixture and has become non-permanently immobilized within said fabricated nanofibrous tubular structure as a consequence of said electrospinning perfusion process, said non-permanently immobilized active agent being released from said nanofibrous tubular structure and delivered in-situ into the surrounding environment as mobile active agent after said nanofibrous tubular structure takes up fluid.

6. An agent-releasing textile useful for the making of a medical article or device, said agent-releasing textile comprising:
a nanofibrous composite material comprised of at least one biodurable synthetic substance and fabricated as a flat sheet fabric via an electrospinning perfusion process, said nanofibrous flat sheet fabric having a determinable length, width, and depth and being biocompatible with the tissues and organs of a living subject; and
at least one pre-chosen biologically active agent having recognized and characteristic mediating properties which has been combined with said biodurable synthetic substance in liquid admixture and has become non-permanently immobilized within said fabricated flat sheet fabric as a consequence of said electrospinning perfusion process, said non-permanently immobilized active agent being released from said nanofibrous flat sheet fabric and delivered in-situ into the surrounding environment as mobile active agent after said nanofibrous flat sheet fabric takes up fluid.

7. The agent-releasing textile recited in claim 5 or 6 wherein said biodurable synthetic substance is a polymer selected from the group consisting of polyethylene terephthalate, nylon, polyurethane, polyglycolic acid, polyamides, polytetrafluoroethylene, polyesters, and mixtures of these substances.

8. The agent-releasing textile recited in claim 5 or 6 wherein said biodurable synthetic substance is a compound selected from the group consisting of an acetate, triacetate, acrylic, acrylonitile, aramid, modacrylic, olefin, propylene, ethylene, and saran.

9. The agent-releasing textile recited in claim 5 or 6 wherein said biologically active agent is an antimicrobial.

10. The agent-releasing textile recited in claim 5 or 6 wherein said biologically active agent is selected from the group consisting of antibiotics, antiseptic, anti-fungals, antimicrobial peptide, analgesic and/or antivirals

11. The agent-releasing textile recited in claim 5 or 6 wherein said biologically active agent is selected from the group consisting of proteins and proteinaceous matter.

12. The agent-releasing textile recited in claim 5 or 6 wherein said biologically active agent is a genetic material.

13. The agent-releasing textile recited in claim 5 or 6 wherein said biologically active agent is selected from the group consisting of pharmacologically active and physiologically active compositions.

14. An electrospinning perfusion method for fabricating a flat sheet textile fabric, said method comprising the steps of:
erecting an electrospinning perfusion assembly comprised of a rotating flat surface which can be set at a selected rotation speed, at least one perfusion instrument which can be set at a specified liquid flow rate, and an electrical coupling for controlling and coordinating the actions of said perfusion instrument upon said rotating flat surface;
preparing a fluid mixture comprised of at least one biodurable synthetic substance and an organic liquid carrier;
introducing said prepared fluid mixture to said perfusion instrument of said assembly;
perfusing said fluid admixture onto said rotating flat surface for a predetermined time such that a nanofibrous flat sheet textile fabric is fabricated, wherein said nanofibrous flat sheet textile fabric has a determinable length, width, and depth and is biocompatible with the tissues and organs of a living subject.

15. An electrospinning perfusion method for fabricating an agent-releasing textile fabric, said method comprising the steps of:
erecting an electrospinning perfusion assembly comprised of a rotating flat surface which can be set at a selected rotation speed, at least one perfusion instrument which can be set at a specified liquid flow rate, and an electrical coupling for controlling and coordinating the actions of said perfusion instrument upon said rotating flat surface;
preparing a fluid mixture comprised of at least one biodurable synthetic substance, at least one pre-chosen biologically active agent having recognized and characteristic mediating properties, and an organic liquid carrier;
introducing said prepared fluid mixture to said perfusion instrument of said assembly;
perfusing said fluid admixture onto said rotating flat surface for a predetermined time such that a nanofibrous flat sheet textile fabric is fabricated, wherein said nanofibrous flat sheet textile fabric has a determinable length, width, and depth and said biologically active agent has become non-permanently immobilized into the fibers of said fabricated nanofibrous flat sheet textile fabric as a consequence of said perfusion, said non-permanently immobilized active agent being released from said nanofibrous flat sheet textile fabric and delivered in-situ into the surrounding environment as mobile active agent after said flat sheet fabric takes up fluid.

16. An electrospinning perfusion method for fabricating an agent-releasing textile fabric, said method comprising the steps of:
erecting an electrospinning perfusion assembly comprised of a rotating mandrel which can be set at a selected rotation speed, at least one perfusion instrument which can be set at a specified liquid flow rate, and an electrical coupling for controlling and coordinating the actions of said perfusion instrument upon said rotating mandrel;
preparing a fluid mixture comprised of at least one biodurable synthetic substance, at least one pre-chosen biologically active agent having recognized and characteristic mediating properties, and an organic liquid carrier;
introducing said prepared fluid mixture to said perfusion instrument of said assembly;
perfusing said fluid admixture onto said rotating mandrel for a predetermined time such that a nanofibrous tubular textile is fabricated, wherein said nanofibrous tubular textile has determinable inner and outer wall diameters, two open ends, and an internal lumen, and is biocompatible for the conveyance of fluid through its internal lumen, and said biologically active agent has become non-permanently immobilized within said nanofibrous tubular textile as a consequence of said perfusion, said non-permanently immobilized active agent being released from said nanofibrous tubular textile and delivered in-situ into the surrounding environment as mobile active agent after said tubular textile takes up fluid.

17. The electrospinning perfusion method recited in claim 14, 15, or 16 wherein said organic liquid carrier of said fluid mixture is selected from the group consisting of hexafluoroisopropanol, dimethylformamide, dimethylsulfoxide, acetonitrile, acetone, hexamethylphosphoric triamide, N,N-diethylacetamine, 4-methylmorpholine-N-oxide monohydrate and N-methylpyrrolidinone.

18. The electrospinning perfusion method recited in claim 14, 15, or 16 wherein said biodurable synthetic substance of said fluid mixture is a polymer.

19. The electrospinning perfusion method recited in claim 14, 15 or 16 wherein said biodurable synthetic substance of said fluid mixture is a polymer selected from the group consisting of polyethylene terephthalate, nylon, polyurethane, polyglycolic acid, polyamides, polytetrafluoroethylene, polyesters, and mixtures of these substances.

20. The electrospinning perfusion method recited in claim 14, 15, or 16 wherein said biodurable synthetic substance of said fluid mixture is a compound selected from the group consisting of an acetate, triacetate, acrylic, acrylonitile, aramid, modacrylic, olefin, propylene, ethylene, and saran.

21. The electrospinning perfusion method recited in claim 14, 15, or 16 wherein said biologically active agent is an antimicrobial.

22. The electrospinning perfusion method recited in claim 14, 15, or 16 wherein said biologically active agent is selected from the group consisting of antibiotics, antiseptics, anti-fungals, antimicrobial peptides and antivirals.

23. The electrospinning perfusion method recited in claim 14, 15, or 16 wherein said biologically active agent is selected from the group consisting of proteins and proteinaceous matter.

24. The electrospinning perfusion method recited in claim 14, 15, or 16 wherein said biologically active agent is a genetic material.

25. The electrospinning perfusion method recited in claim 14, 15, or 16 wherein said biologically active agent is selected from the group consisting of pharmacologically active and physiologically active compositions.
